(19) United States
(12) Patent Application Publication
(10) Pub. No.: US 2026/0151634 A1
(43) Pub. Date: Jun. 4, 2026

(54) SYSTEM AND METHOD FOR DETECTING HEMODYNAMICALLY UNSTABLE CARDIAC RHYTHMS

(71) Applicant: Medtronic, Inc., Minneapolis, MN (US)

(72) Inventors: Saul E. GREENHUT, Denver, CO (US); Richard J. O'BRIEN, Hugo, MN (US); Randal C. SCHULHAUSER, Phoenix, AZ (US)

(21) Appl. No.: 19/122,719

(22) PCT Filed: Oct. 10, 2023
(86) PCT No.: PCT/IB2023/060193

§ 371 Date: Apr. 18, 2025

Related U.S. Application Data
(60) Provisional application No. 63/381,126, filed on Oct. 26, 2022.

(51) Int. Cl. A61N 1/365; A61N 1/02; A61N 1/372; A61N 1/39

(52) U.S. Cl. CPC A61N 1/36585; A61N 1/025; A61N 1/36514; A61N 1/36592; A61N 1/3925; A61N 1/39622; A61N 1/37211

(57) ABSTRACT

A medical device system is configured to sense electrical brain activity and sense electrical cardiac activity. The system may detect a concerning heart rate from the electrical cardiac activity and determine that the concerning heart rate is a hemodynamically unstable cardiac rhythm based on the sensed electrical brain activity. The medical device system may generate an output for providing a response to determining that the concerning heart rate is a hemodynamically unstable rhythm.

### TECHNICAL FIELD

This application claims the benefit of U.S. Provisional Patent Application Ser. No. 63/381,126, filed Oct. 26, 2022, the entire content of which is incorporated herein by reference.

The disclosure relates generally to a medical device system and method for detecting a hemodynamically unstable cardiac rhythm based on electrical cardiac activity and electrical brain activity.

### BACKGROUND

Medical devices may sense electrophysiological signals from the heart, brain, nerve, muscle or other tissue. Such devices may be implantable, partially implantable, wearable or external devices using implantable and/or surface (skin) electrodes for sensing the electrophysiological signals. In some cases, such devices may be configured to deliver a therapy based on the sensed electrophysiological signals. For example, implantable or external cardiac pacemakers, cardioverter defibrillators, cardiac monitors and the like, sense cardiac electrical signals from a patient's heart. A cardiac pacemaker or cardioverter defibrillator may sense cardiac electrical signals from the heart and deliver electrical stimulation therapies to the heart using electrodes carried by a transvenous medical electrical lead, a non-transvenous medical electrical lead and/or leadless electrodes coupled directly to the housing of the medical device.

The electrical stimulation therapies may include signals such as pacing pulses or cardioversion/defibrillation shocks. In some cases, a medical device may sense cardiac event signals attendant to the intrinsic or pacing-evoked depolarizations of the heart and control delivery of stimulation signals to the heart based on sensed cardiac event signals. Upon detection of an abnormal rhythm based on the sensed cardiac event signals (or absence thereof), such as bradycardia, tachycardia or fibrillation, an appropriate electrical stimulation signal or signals may be delivered to restore or maintain a more normal rhythm of the heart. For example, an implantable cardioverter defibrillator (ICD) may deliver pacing pulses to the heart of the patient upon detecting bradycardia or tachycardia or deliver cardioversion/defibrillation (CV/DF) shocks to the heart upon detecting tachycardia or fibrillation.

### SUMMARY

In general, the disclosure is directed to a medical device system and method for discriminating between hemodynamically unstable (HU) and hemodynamically stable (HS) cardiac rhythms. A medical device system operating according to the techniques disclosed herein senses electrophysiology signals, e.g., electrical brain activity and electrical cardiac activity. In some examples the medical device system includes two or more implantable medical devices each configured to sense electrophysiology signals and communicate wirelessly to cooperatively detect a concerning heart rate and determine if the concerning heart rate is HU. A concerning heart rate may be determined to be HU when one or more metrics determined from the sensed electrical brain activity meet brain ischemia criteria. The one or more metrics may be representative of the amplitude and/or frequency of the electrical brain activity and may be determined in the time domain and/or the frequency domain.

The concerning heart rate may be a fast heart rate associated with a tachycardia or fibrillation, which may be hemodynamically stable or unstable. In some instances, the concerning heart rate may be a relatively slow or irregular heart rate, e.g., associated with bradycardia or an irregularly conducted heart rhythm. Based on whether the concerning heart rate is HU or HS, the medical device system may select a response, which may include delivering one or more cardiac electrical stimulation therapies, withholding a cardiac electrical stimulation therapy, and/or transmitting an alarm, notification or other communication when the concerning heart rate is determined to be HU.

In one example, the disclosure provides a medical device system including sensing circuitry configured to sense electrical brain activity and electrical cardiac activity. The medical device system includes processing circuitry configured to detect a concerning heart rate from the electrical cardiac activity and may determine that the concerning heart rate is a hemodynamically unstable cardiac rhythm based on the sensed electrical brain activity. The processing circuitry may generate an output for providing a response to determining that the concerning heart rate is a hemodynamically unstable cardiac rhythm. The medical device system may include a memory configured to store the output generated by the processing circuitry.

In another example, the disclosure provides a method including sensing electrical brain activity, sensing electrical cardiac activity, and detecting a concerning heart rate from the electrical cardiac activity by processing circuitry of the medical device system. The method may include determining that the concerning heart rate is a hemodynamically unstable cardiac rhythm based on the sensed electrical brain activity and generating an output for providing a response to determining that the concerning heart rate is a hemodynamically unstable rhythm. The method may include storing the generated output in a memory of the processing circuitry.

In another example, the disclosure provides a non-transitory computer-readable medium storing instructions which, when executed by processing circuitry of a medical device system, cause the system to sense electrical brain activity, sense electrical cardiac activity, detect a concerning heart rate from the electrical cardiac activity and determine that the concerning heart rate is a hemodynamically unstable cardiac rhythm based on the sensed electrical brain activity. The instructions may further cause the medical device system to generate an output for providing a response to determining that the concerning heart rate is a hemodynamically unstable rhythm. The instructions may cause the system to store the generated output in a memory of the medical device system.

Further disclosed herein is the subject matter of the following examples:

Example 1. A medical device system including sensing circuitry configured to sense electrical brain activity and electrical cardiac activity and including processing circuitry configured to detect a concerning heart rate from the sensed electrical cardiac activity. The processing circuitry may be further configured to determine that the concerning heart rate is a hemodynamically unstable cardiac rhythm based on the sensed electrical brain activity and generate an output for providing a response to determining that the concerning heart rate is a hemodynamically unstable cardiac rhythm. The medical device system may include a memory configured to store the output generated by the processing circuitry.

Example 2. The medical device system of example 1 further including a therapy delivery circuit configured to deliver an electrical stimulation therapy. The control circuit may be further configured to generate the output by generating a therapy control signal, and the therapy delivery circuit can be configured to deliver an electrical stimulation therapy according to the therapy control signal.

Example 3. The medical device system of any of examples 1-2 wherein the processing circuitry is further configured to determine at least one amplitude metric from the electrical brain activity and compare the amplitude metric to brain ischemia criteria. The processing circuitry may determine that the concerning heart rate is a hemodynamically unstable cardiac rhythm in response to the amplitude metric meeting the brain ischemia criteria.

Example 4. The medical device system of any of examples 1-3 wherein the processing circuitry is further configured to determine at least one frequency metric from the electrical brain activity, compare the frequency metric to brain ischemia criteria and determine that the concerning heart rate is a hemodynamically unstable cardiac rhythm in response to the frequency metric meeting the brain ischemia criteria.

Example 5. The medical device system of example 4, wherein the processing circuitry is further configured to determine the at least one frequency metric from the electrical brain activity by determining at least one of: a dominant frequency of the electrical brain activity; a number of oscillations of the electrical brain activity during a time interval; and/or an amplitude of a signal derivative of the electrical brain activity. The processing circuitry may further determine that the frequency metric meets brain ischemia criteria; and determine that the concerning heart rate is a hemodynamically unstable cardiac rhythm in response to the frequency metric meeting the brain ischemia criteria.

Example 6. The medical device system of any of examples 1-5 wherein the processing circuitry is further configured to determine at least one metric of the electrical brain activity and determine a stage of brain ischemia from a plurality of stages of brain ischemia based on the at least one metric. The processing circuitry may determine that the concerning heart rate is a hemodynamically unstable cardiac rhythm based on the determined stage of brain ischemia.

Example 7. The medical device system of any of examples 1-6 wherein the processing circuitry is further configured to determine a metric from the electrical brain activity for each of a plurality of time intervals and determine that a change in the metric determined for each of the plurality of time intervals meets brain ischemia criteria. The processing circuitry may determine that the concerning heart rate is a hemodynamically unstable cardiac rhythm based on the change in the metric meeting brain ischemia criteria.

Example 8. The medical device system of example 7 wherein the processing circuitry is further configured to determine that the change in the metric meets the brain ischemia criteria by at least determining that the change in the metric is within a threshold time interval of an onset of the concerning heart rate.

Example 9. The medical device system of any of examples 1-8 wherein the processing circuitry is further configured to detect the concerning heart rate by detecting a heart rate that is faster than a threshold rate.

Example 10. The medical device system of example 9 further comprising a therapy delivery circuit configured to deliver a cardioversion/defibrillation shock. The processing circuitry can be further configured to determine that the sensed electrical brain activity meets severe brain ischemia criteria and control the therapy delivery circuit to abort a cardioversion/defibrillation shock in response to determining that the sensed electrical brain activity meets the severe brain ischemia criteria.

Example 11. The medical device system of any of examples 1-10 further including a therapy delivery circuit configured to generate a cardiac electrical stimulation therapy. The processing circuitry can be further configured to detect a monomorphic ventricular tachyarrhythmia associated with the concerning heart rate based on the sensed cardiac electrical activity and control the therapy delivery circuit to deliver a first anti-tachycardia pacing therapy in response to detecting the monomorphic ventricular tachyarrhythmia. the processing circuitry may be configured to determine that the monomorphic ventricular tachyarrhythmia is not terminated by the first anti-tachycardia pacing therapy, and, in response to determining that the monomorphic ventricular tachyarrhythmia is not terminated, determine at least one metric from the sensed electrical brain activity. The processing circuitry may be further configured to determine that the concerning heart rate is a hemodynamically unstable cardiac rhythm based on the sensed electrical brain activity by determining that the monomorphic ventricular tachyarrhythmia is a hemodynamically unstable cardiac rhythm based on the at least one metric.

Example 12. The medical device system of example 11 wherein the processing circuitry is further configured to generate the output for providing the response by generating a cardioversion/defibrillation shock therapy signal.

Example 13. The medical device system of any of examples 2-12 wherein the processing circuitry is further configured to detect a polymorphic ventricular tachycardia or ventricular fibrillation associated with the concerning heart rate and control the therapy delivery circuit to deliver a cardioversion/defibrillation shock therapy in response to detecting the polymorphic ventricular tachycardia or ventricular fibrillation without determining that the polymorphic ventricular tachycardia or ventricular fibrillation is hemodynamically unstable based on the sensed brain activity.

Example 14. The medical device system of any of examples 1-8 wherein the processing circuitry is further configured to detect the concerning heart rate by detecting one of a heart rate slower than a threshold rate or heart rate that meets variable heart rate criteria.

Example 15. The medical device system of example 14 wherein the processing circuitry is further configured to generate the output by generating a cardiac pacing delivery signal.

Example 16. The medical device system of any of examples 14-15 wherein the processing circuitry is further configured to generate the output by increasing a lower pacing rate.

Example 17. The medical device system of any of examples 1-16 wherein the processing circuitry is further configured to detect a normal sinus rhythm from the electrical cardiac activity and establish brain ischemia criteria based on electrical brain activity sensed during the detected normal sinus rhythm. The processing circuitry further configured to determine that the concerning heart rate is hemodynamically unstable based on the sensed electrical brain activity meeting the brain ischemia criteria.

Example 18. The medical device system of any of examples 1-17 further comprising a communication circuit. The processing circuitry is further configured to generate the output for providing the response by generating an alert signal, and the communication circuit is configured to transmit the alert signal.

Example 19. The medical device system of any of examples 1-18 further comprising at least one sensor configured to sense a sensor signal received by the processing circuitry.

The processing circuitry is further configured to detect sleep based on at least one of a time of day or the sensor signal and determine that the concerning heart rate being hemodynamically unstable is indeterminate based on the sensed electrical brain activity in response to detecting sleep.

Example 20. The medical device system of any of examples 1-18 further comprising at least one sensor configured to sense a sensor signal received by the processing circuitry. The processing circuitry being further configured to detect sleep based on at least one of a time of day or the sensor signal, generate an arousal signal and detect brain ischemia based on the electrical brain activity sensed after the arousal signal. The processing circuitry being configured to determine that the concerning heart rate is a hemodynamically unstable cardiac rhythm based on detecting the brain ischemia based on the electrical brain activity sensed after the arousal signal.

Example 21. The medical device system of any of examples 1-20 wherein the processing circuitry is further configured to detect termination of the concerning heart rate. The processing circuitry may determine that the sensed electrical brain activity still meets brain ischemia criteria after detecting termination of the concerning heart rate and adjust a control parameter used in detecting the concerning heart rate from the sensed electrical cardiac activity in response to determining that the sensed electrical brain activity still meets brain ischemia criteria.

Example 22. The medical device system of any of examples 1-21 wherein the processing circuitry is further configured to detect a hemodynamically stable cardiac rhythm based on the sensed electrical cardiac activity and the sensed electrical brain activity and generate a second output in response to detecting the hemodynamically stable cardiac rhythm. The memory is configured to store the second output.

Example 23. The medical device system of example 22 further comprising a therapy delivery circuit configured to deliver a cardioversion/defibrillation shock therapy. The processing circuitry is further configured to generate the second output by generating a therapy control signal for withholding the cardioversion/defibrillation shock therapy.

Example 24. A method comprising sensing electrical brain activity by sensing circuitry of a medical device system, sensing electrical cardiac activity by sensing circuitry of the medical device system, detecting a concerning heart rate from the sensed electrical cardiac activity by processing circuitry of the medical device system and determining that the concerning heart rate is a hemodynamically unstable cardiac rhythm based on the sensed electrical brain activity. The method may further include generating an output by the processing circuitry for providing a response to determining that the concerning heart rate is a hemodynamically unstable rhythm and may include storing the generated output in a memory of the medical device system.

Example 25. The method of example 24 further comprising generating the output by generating a therapy control signal and delivering an electrical stimulation therapy according to the therapy control signal.

Example 26. The method of any of examples 24-25 further comprising determining at least one amplitude metric from the electrical brain activity, comparing the amplitude metric to brain ischemia criteria and determining that the concerning heart rate is a hemodynamically unstable cardiac rhythm in response to the amplitude metric meeting the brain ischemia criteria.

Example 27. The method of any of examples 24-26 further comprising determining at least one frequency metric from the electrical brain activity, comparing the frequency metric to brain ischemia criteria and determining that the concerning heart rate is a hemodynamically unstable cardiac rhythm in response to the frequency metric meeting the brain ischemia criteria.

Example 28. The method of example 27 further comprising determining the at least one frequency metric from the electrical brain activity by determining at least one of: a dominant frequency of the electrical brain activity, a number of oscillations of the electrical brain activity during a time interval and/or an amplitude of a signal derivative of the electrical brain activity. The method may further include determining that the frequency metric meets brain ischemia criteria and determining that the concerning heart rate is a hemodynamically unstable cardiac rhythm in response to the frequency metric meeting the brain ischemia criteria.

Example 29. The method of any of examples 24-28 further comprising determining at least one metric of the electrical brain activity, determining a stage of brain ischemia from a plurality of stages of brain ischemia based on the at least one metric and determining that the concerning heart rate is a hemodynamically unstable cardiac rhythm based on the determined stage of brain ischemia.

Example 30. The method of any of examples 24-29 further comprising determining a metric from the electrical brain activity for each of a plurality of time intervals, determining that a change in the metric determined for each of the plurality of time intervals meets brain ischemia criteria, and determining that the concerning heart rate is a hemodynamically unstable cardiac rhythm based on the change in the metric meeting brain ischemia criteria.

Example 31. The method of example 30 further comprising determining that the change in the metric meets the brain ischemia criteria by at least determining that the change in the metric is within a threshold time interval of an onset of the concerning heart rate.

Example 32. The method of any of examples 24-31 further comprising detecting the concerning heart rate by detecting a heart rate that is faster than a threshold rate.

Example 33. The method of example 32 further comprising determining that the sensed electrical brain activity meets severe brain ischemia criteria and aborting a cardioversion/defibrillation shock in response to determining that the sensed electrical brain activity meets the severe brain ischemia criteria.

Example 34. The method of any of examples 25-33 further comprising detecting a monomorphic ventricular tachyarrhythmia associated with the concerning heart rate based on the sensed cardiac electrical activity and delivering a first anti-tachycardia pacing therapy in response to detecting the monomorphic ventricular tachyarrhythmia. The method may further include determining that the monomorphic ventricular tachyarrhythmia is not terminated by the first anti-tachycardia pacing therapy. In response to determining that the monomorphic ventricular tachyarrhythmia is not terminated, determining at least one metric from the sensed electrical brain activity and determining that the concerning heart rate is a hemodynamically unstable cardiac rhythm based on the sensed electrical brain activity by determining that the monomorphic ventricular tachyarrhythmia is a hemodynamically unstable cardiac rhythm based on the at least one metric.

Example 35. The method of example 34 further comprising generating the output for providing the response by generating a cardioversion/defibrillation shock therapy signal.

Example 36. The method of any of examples 25-35, further comprising detecting a polymorphic ventricular tachycardia or ventricular fibrillation associated with the concerning heart rate and delivering a cardioversion/defibrillation shock therapy in response to detecting the polymorphic ventricular tachycardia or ventricular fibrillation without determining that the polymorphic ventricular tachycardia or ventricular fibrillation is hemodynamically unstable based on the sensed brain activity.

Example 37. The method of any of examples 24-31 further comprising detecting the concerning heart rate by detecting one of a heart rate slower than a threshold rate or a heart rate that meets variable heart rate criteria.

Example 38. The method of example 37 further comprising generating the output by generating a cardiac pacing signal.

Example 39. The method of any of examples 35-36 further comprising generating the output by increasing a lower pacing rate.

Example 40. The method of any of examples 24-39 further comprising detecting a normal sinus rhythm from the electrical cardiac activity and establishing brain ischemia criteria based on electrical brain activity sensed during the detected normal sinus rhythm. The method further including determining that the concerning heart rate is hemodynamically unstable based on the sensed electrical brain activity meeting the brain ischemia criteria.

Example 41. The method of any of examples 24-40 further comprising generating the output for providing the response by generating an alert signal and transmitting the alert signal.

Example 42. The method of any of examples 24-41, further comprising receiving at least one sensor signal, detecting sleep based on at least one of a time of day or the sensor signal and determining that the concerning heart rate being a hemodynamically unstable cardiac rhythm is indeterminate based on the sensed electrical brain activity in response to detecting sleep.

Example 43. The method of any of examples 24-41 further comprising receiving at least one sensor signal, detecting sleep based on at least one of a time of day or the sensor signal, generating an arousal signal and detecting brain ischemia based on the sensed electrical brain activity after the arousal signal. The method further including determining that the concerning heart rate is a hemodynamically unstable cardiac rhythm based on detecting the brain ischemia based on the electrical brain activity sensed after the arousal signal.

Example 44. The method of any of examples 24-43 further comprising detecting termination of the concerning heart rate, determining that the sensed electrical brain activity still meets brain ischemia criteria after detecting termination of the concerning heart rate and adjusting a control parameter used in detecting the concerning heart rate from the sensed electrical cardiac activity in response to determining that the sensed electrical brain activity still meets brain ischemia criteria.

Example 45. The method of any of examples 24-44 further comprising detecting a hemodynamically stable cardiac rhythm based on the sensed electrical cardiac activity and the sensed electrical brain activity, generating a second output in response to detecting the hemodynamically stable cardiac rhythm and storing the second output in the memory.

Example 46. The method of example 45 further comprising generating the second output by generating a therapy control signal for withholding a cardioversion/defibrillation shock therapy.

Example 47. Non-transitory computer-readable storage media storing instructions which, when executed by processing circuitry of a medical device system, cause the system to sense electrical brain activity, sense electrical cardiac activity, detect a concerning heart rate from the sensed electrical cardiac activity, and determine that the concerning heart rate is a hemodynamically unstable cardiac rhythm based on the sensed electrical brain activity. The instructions may further cause the medical device system to generate an output for providing a response to determining that the concerning heart rate is a hemodynamically unstable rhythm and store the generated output in a memory of the medical device system.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual diagram of multiple implantable medical devices (IMDs) that may be co-implanted in a medical device system configured to sense electrical cardiac activity and electrical brain activity for discriminating between an HU cardiac rhythm and an HS cardiac rhythm.

FIG. 2 is a conceptual diagram of another medical device system configured to sense electrical cardiac activity and electrical brain activity for detecting an HU cardiac rhythm according to another example.

FIG. 3 is a conceptual diagram of a medical device system configured to sense electrical cardiac activity and electrical brain activity for detecting an HU cardiac rhythm according to yet another example.

FIG. 4 is a conceptual diagram of an IMD configured to sense cardiac electrical signals and deliver cardiac electrical stimulation therapies according to one example.

FIG. 5 is a conceptual diagram of an electrocochleography (EEG) sensor according to one example.

FIG. 6 is a flow chart of a method that may be performed by a medical device system for discriminating between HU and HS cardiac rhythms according to some examples.

FIG. 7 is a diagram of various examples of EEG signals that may be sensed by the EEG sensor of FIG. 5 during varying stages of brain ischemia.

FIG. 8 is a flow chart of a method for detecting brain ischemia for use in classifying a cardiac rhythm as being HU or HS according to some examples.

FIG. 9 is a flow chart of a method that may be performed by a medical device system for detecting a tachyarrhythmia and selecting a therapy response based on a determination of whether the tachyarrhythmia is HU or HS according to some examples.

FIG. 10 is a flow chart of a method that may be performed by a medical device system for detecting a tachyarrhythmia and selecting a therapy response according to another example.

FIG. 11 is a flow chart of a method for detecting HU and HS cardiac rhythms according to another example.

### DETAILED DESCRIPTION

In general, this disclosure describes a medical device system and techniques for detecting hemodynamically unstable (HU) cardiac rhythms based on processing and analysis of cardiac electrical signals and brain electrical signals. Cardiac electrical activity may be sensed and analyzed by a medical device for detecting a concerning heart rate, which may be a fast heart rate arising from tachycardia or fibrillation or a slow heart rate, e.g., arising from bradycardia or irregularly conducted heart beats that cause an irregular or variable heart rate. A slow or irregular heart rate may be concerning in some patients that experience various types of syncope, hypotension or other conditions. In some instances, a slow heart rate may be falsely detected when cardiac event signals (e.g., P-waves attendant to atrial depolarizations or R-waves attendant to ventricular depolarizations) in the cardiac electrical signal are undersensed during tachycardia or fibrillation, which may be HU. The analysis of the cardiac electrical signals may include discriminating between types of tachyarrhythmias such a supraventricular tachyarrhythmia (SVT), ventricular tachycardia (VT) and ventricular fibrillation (VF).

When a concerning heart rate is detected, the discrimination between an HS and an HU cardiac rhythm may be determined based on analysis electrical brain activity, e.g., an EEG, which may be sensed and analyzed by a second medical device in some medical device systems. Various examples of combinations of IMDs are described herein that can be configured to process and analyze cardiac electrical signals, which may be sensed by one IMD, and brain electrical signals, e.g., EEG signals, which may be sensed by a second IMD, with the two IMDs in communication with each other (directly or indirectly via one or more relay devices) for cooperatively detecting HU cardiac rhythms. The cardiac electrical activity may be sensed as electrocardiogram (ECG) signal(s) using electrodes implanted away from the heart and/or cardiac electrogram (EGM) signals(s) sensed from electrodes implanted in or on the heart.

In some examples, at least one IMD configured to sense the cardiac electrical activity may be further configured to deliver a cardiac electrical stimulation therapy. The cardiac electrical stimulation therapy may be delivered to treat bradycardia (when a slow heart rate is detected that is determined to be HU) or to treat tachyarrhythmias (when a fast heart is detected). According to the techniques disclosed herein, a therapy response to a detected cardiac rhythm may be selected based on whether the detected cardiac rhythm is HS or HU. Cardiac electrical stimulation therapies may include bradycardia pacing, anti-tachyarrhythmia pacing (ATP) and/or cardioversion/defibrillation (CV/DF) shocks. In some examples, electrical stimulation therapies may include neurostimulation therapies, such as spinal cord stimulation or vagal nerve stimulation to alter the autonomic tone to promote or restore hemodynamic stability. Therapy delivery decisions, e.g., whether to deliver a therapy or not or selection from among multiple available therapies, e.g., ATP or a CV/DF shock, may be made by the medical device system based on the determination of an HU or HS cardiac rhythm in various examples.

In some illustrative examples presented herein, the IMD configured to sense cardiac electrical signals may be a pacemaker or implantable cardioverter defibrillator (ICD) that is configured to sense cardiac electrical signals and deliver cardiac electrical stimulation pulses for cardiac pacing and/or CV/DF shock delivery. The pacemaker or ICD may be coupled to one or more transvenous or non-transvenous leads for carrying electrodes for sensing cardiac electrical activity and delivering electrical stimulation therapy. For example, the pacemaker or ICD may be coupled to an “extra-cardiovascular” lead, referring to a lead that positions electrodes outside the blood vessels, heart, and pericardium surrounding the heart of a patient. An extra-cardiovascular lead may also be referred to as a “non-transvenous” lead. Implantable electrodes carried by extra-cardiovascular leads, for example, may be positioned extra-thoracically (outside the ribcage and sternum, e.g., subcutaneously or submuscularly) or intra-thoracically (e.g., beneath the ribcage or sternum, sometimes referred to as a “sub-sternal” position). The electrodes used for sensing cardiac electrical activity and/or the electrodes used for delivering cardiac electrical stimulation therapies may or may not be in intimate contact with myocardial tissue. For instance, electrodes carried by one or more non-transvenous leads can be implanted within the pericardium and coupled to an IMD in a medical device system configured to operate according to the techniques disclosed herein.

In other examples, the pacemaker or ICD may be coupled to a transvenous lead that positions electrodes within a blood vessel, which may remain outside the heart in an “extra-cardiac” location or be advanced to position electrodes within a heart chamber, e.g., in or along the endocardium. In some cases, a transvenous medical lead may be advanced along a venous pathway to position electrodes in an extra-cardiac location within the internal thoracic vein (ITV), an intercostal vein, the superior epigastric vein, or the azygos, hemiazygos, or accessory hemiazygos veins, as examples. In still other examples, one or more transvenous leads may be advanced to position electrodes within the heart, e.g., within an atrial and/or ventricular heart chamber.

In some examples, two or more IMDs may be co-implanted in a patient in a medical device system that is capable of sensing at least one cardiac electrical signal (for sensing electrical cardiac activity) and at least one EEG signal (for sensing electrical brain activity). At least one implanted device configured to sense a cardiac electrical signal may be capable of wireless communication with a second implanted device configured to sense an EEG signal for cooperatively determining whether a cardiac rhythm is either an HU cardiac rhythm or a HS cardiac rhythm. The two devices may be monitoring-only devices, without therapy delivery capability. In other examples, at least one device may be capable of delivering a therapy for treating a patient condition. For example, at least one IMD configured to sense a cardiac electrical signal may be capable of delivering a therapy in response to detecting a tachyarrhythmia. The therapy selected for delivery can be selected based on whether the detected tachyarrhythmia is HU or HS using the techniques disclosed herein. In some examples, one or more of the co-implanted devices may be capable of delivering other therapies for treating other patient conditions. For example, the IMD configured to sense an EEG signal may be configured to deliver neurostimulation therapy, deep brain stimulation, drug delivery, or other therapy for treating a patient condition other than a cardiac arrhythmia. Though in some cases, an IMD configured to sense electrical brain activity may be positioned for delivering vagal nerve stimulation, spinal cord stimulation or other neurostimulation for altering the patient's autonomic tone, which may be delivered in some examples as a therapy response to a detected cardiac rhythm that is HU.

Furthermore, it is recognized that one or more IMDs may be configured to monitor electrical cardiac activity and electrical brain activity for detecting and discriminating between HS and HU cardiac rhythms, the one or more IMDs configured to detect HS and HU cardiac rhythms, may be in communication with a third IMD configured to deliver a therapy. The determination of a HS or HU cardiac rhythm may be transmitted to a third IMD configured to select and deliver an appropriate therapy based on the determination of a HS or HU cardiac rhythm.

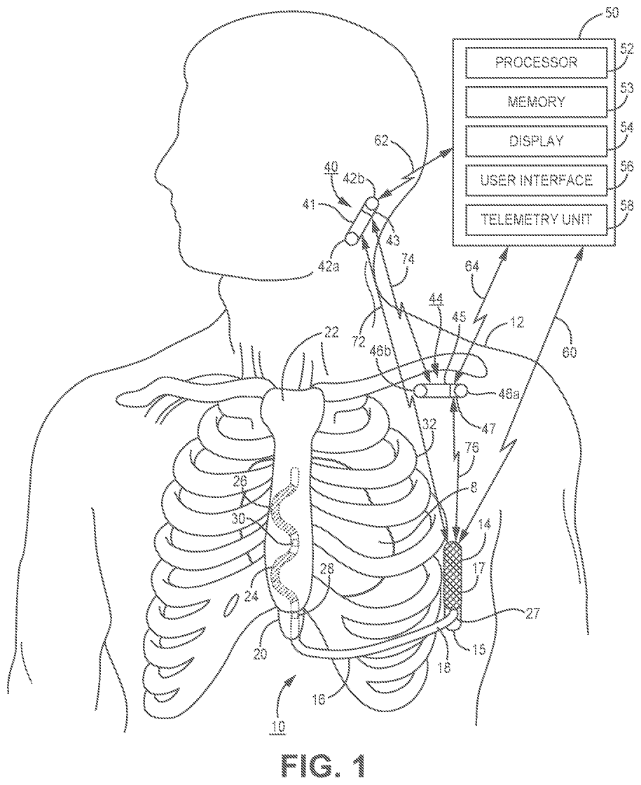

The IMDs shown and described below in conjunction with FIGS. 1-3 are illustrative examples of IMDs that may be co-implanted in a patient and configured to perform techniques disclosed herein, with no limitation intended. It is to be understood that the techniques disclosed herein are not limited to being implemented in a medical device system that includes only implantable devices configured to detect HU and HS cardiac rhythms. For example, the devices for sensing an EEG signal and a cardiac electrical signal for discriminating between HU and HS cardiac rhythms may include an external medical device. One or more medical devices included in a medical device system configured to perform the techniques disclosed herein may be an external device, e.g., a wearable device having electrodes for sensing electrical brain activity and/or electrical cardiac activity, such as an external EEG monitor, external ECG monitor, smart watch, fitness tracker or the like. In some examples, a medical device system configured to perform the techniques disclosed herein may include a partially implantable device which may include one or more implantable components, e.g., electrodes carried by a transcutaneous lead or conductors, coupled to one or more external components, e.g., an external housing enclosing circuitry coupled to implanted electrodes.

FIG. 1 is a conceptual diagram of multiple IMDs that may be co-implanted in a medical device system 10 configured to sense electrical cardiac activity and electrical brain activity for detecting and discriminating between HU cardiac rhythms and HS cardiac rhythms. In this example, patient 12 is shown implanted with an ICD 14 connected to a non-transvenous, extra-cardiovascular electrical stimulation and sensing lead 16. ICD 14 may be configured to sense at least one cardiac electrical signal, e.g., an ECG signal, for detecting a concerning heart rate. ICD 14 may optionally detect and discriminate between different types of tachyarrhythmia, e.g., SVT, VT and VF when a fast heart rate is detected. Additionally or alternatively, patient 12 may be implanted with a cardiac monitor 44 for sensing a cardiac electrical signal for detecting a concerning heart rate using housing-based electrodes 46a and 46b (collectively electrodes 46). Patient 12 may also be implanted with an EEG sensor 40, which may include housing-based electrodes 42a and 42b (collectively electrodes 42) for sensing electrical brain activity, e.g., an EEG signal.

In some examples, patient 12 may be implanted with cardiac monitor 44 and EEG sensor 40 for detecting HU cardiac rhythms and HS cardiac rhythms without necessarily having automatic therapy delivery capabilities for treating or terminating a tachyarrhythmia. Furthermore, depending on the implant location of EEG sensor 40 or cardiac monitor 44, a single implanted device, e.g., EEG sensor 40 or cardiac monitor 44, may be configured to sense both electrical brain activity and electrical cardiac activity using respective electrodes 42 or 46, for detecting HU and HS cardiac rhythms using the technique disclosed herein. For instance, when EEG sensor 40 is implanted posteriorly near the base of the patient's skull or back of the neck, an electrical signal sensed using electrodes 42 may be processed and analyzed by EEG sensor 40 for determining both brain electrical activity and cardiac electrical activity. EEG sensor 40 may correspond to an implantable medical device as generally disclosed in U.S. Publication No. 2022/0061743 (Christensen, et al.), the content of which is incorporated herein by reference in its entirety. In some examples, a single medical device including one or more electrodes on the housing of the medical device and/or one or more electrodes carried by leads extending from the medical device may be positioned for sensing electrical cardiac activity and electrical brain activity for detecting HU and HS cardiac rhythms according to the techniques disclosed herein.

In other examples, patient 12 may be implanted with ICD 14 in combination with EEG sensor 40, without cardiac monitor 44, for detecting concerning heart rates and delivering cardiac electrical stimulation therapies selected based on a determination of whether the concerning heart rate is HS or HU. In still other examples, patient 12 may be implanted with all three devices, ICD 14, cardiac monitor 44 and EEG sensor 40, for sensing cardiac electrical signals, sensing EEG signals, detecting concerning heart rates, determining if the cardiac rhythm associated with the concerning heart rate is an HU cardiac rhythm or HS cardiac rhythm, and selecting a therapy delivery response based on the HU or HS determination.

ICD 14 includes a housing 15 that forms a hermetic seal that protects internal components of ICD 14. The housing 15 of ICD 14 may be formed of a conductive material, such as titanium or titanium alloy. The housing 15 may function as an electrode (sometimes referred to as a “can” electrode). Housing 15 may be used as an active can electrode for use in delivering CV/DF shocks or other relatively high voltage pulses delivered using a high voltage therapy delivery circuit. In other examples, housing 15 may be available for use in delivering unipolar, low voltage cardiac pacing pulses and/or for sensing cardiac electrical signals in combination with electrodes carried by lead 16. In other instances, the housing 15 of ICD 14 may include a plurality of electrodes on an outer portion of the housing. The outer portion(s) of the housing 15 functioning as an electrode(s) may be coated with a material, such as titanium nitride, e.g., for reducing post-stimulation polarization artifact.

ICD 14 includes a connector assembly 17 (also referred to as a connector block or header) that includes electrical feedthroughs crossing housing 15 to provide electrical connections between conductors extending within the lead body 18 of lead 16 and electronic components included within the housing 15 of ICD 14. As will be described in further detail herein, housing 15 may house one or more processors, memories, transceivers, cardiac electrical signal sensing circuitry, therapy delivery circuitry, power sources and other components for sensing cardiac electrical signals, detecting a cardiac rhythm, and controlling and delivering electrical stimulation pulses to treat an abnormal cardiac rhythm.

Lead 16 is shown in this example as a non-transvenous, extra-cardiovascular lead implanted beneath the sternum 22. Lead 16 includes an elongated lead body 18 having a proximal end 27 that includes a lead connector (not shown) configured to be connected to ICD connector assembly 17 and a distal portion that includes one or more electrodes. In the example illustrated in FIG. 1, the distal portion of lead body 18 includes defibrillation electrodes 24 and 26 and pace/sense electrodes 28 and 30. In some cases, defibrillation electrodes 24 and 26 may together form a defibrillation electrode in that they may be configured to be activated concurrently. Alternatively, defibrillation electrodes 24 and 26 may form separate defibrillation electrodes in which case each of the electrodes 24 and 26 may be activated independently.

Electrodes 24 and 26 (and in some examples housing 15) can be referred to as defibrillation electrodes because they may be utilized, individually or collectively, for delivering high voltage stimulation therapy (e.g., cardioversion or defibrillation shocks). Electrodes 24 and 26 may be elongated coil electrodes and generally have a relatively high surface area for delivering high voltage electrical stimulation pulses compared to pacing and sensing electrodes 28 and 30. However, electrodes 24 and 26 and housing 15 may also be utilized to provide pacing functionality, sensing functionality or both pacing and sensing functionality in addition to or instead of high voltage stimulation therapy. In this sense, the use of the term “defibrillation electrode” herein should not be considered as limiting the electrodes 24 and 26 for use in only high voltage cardioversion/defibrillation shock therapy applications. For example, either of electrodes 24 and 26 may be used as a sensing electrode in a sensing electrode vector for sensing cardiac electrical signals for detecting and discriminating cardiac rhythms and determining a need for an electrical stimulation therapy.

Electrodes 28 and 30 are relatively smaller surface area electrodes which are available for use in sensing electrode vectors for sensing cardiac electrical signals and may be used for delivering relatively low voltage cardiac pacing pulses in some configurations. Electrodes 28 and 30 are referred to herein as “pace/sense electrodes” because they are generally configured for use in low voltage applications, e.g., used as either a cathode or anode for delivery of pacing pulses and/or sensing of cardiac electrical signals, as opposed to delivering high voltage CV/DF shocks. In some instances, electrodes 28 and 30 may provide only pacing functionality, only sensing functionality or both.

ICD 14 may sense cardiac electrical signals corresponding to electrical activity of heart 8 via one or more sensing electrode vectors that include combinations of electrodes 24, 26, 28 and/or 30. In some examples, housing 15 of ICD 14 is used in combination with one or more of electrodes 24, 26, 28 and/or 30 in a sensing electrode vector. In the example illustrated in FIGS. 1A and 1B, electrode 28 is located proximal to defibrillation electrode 24, and electrode 30 is located between defibrillation electrodes 24 and 26. One, two or more pace/sense electrodes may be carried by lead body 18. For instance, a third pace/sense electrode may be located distal to defibrillation electrode 26 in some examples. In some cases, lead body 18 may only carry relatively larger surface area defibrillation electrodes without smaller pace/sense electrodes.

Electrodes 28 and 30 are illustrated as ring electrodes; however, electrodes 28 and 30 may comprise any of a number of different types of electrodes, including ring electrodes, short coil electrodes, hemispherical electrodes, directional electrodes, segmented electrodes, or the like. Electrodes 28 and 30 may be positioned at other locations along lead body 18 and are not limited to the positions shown. In other examples, lead 16 may include fewer or more pace/sense electrodes and/or defibrillation electrodes than the example shown here.

Lead 16 may extend subcutaneously or submuscularly over the ribcage 32 medially from the connector assembly 27 of ICD 14 toward a center of the torso of patient 12, e.g., toward xiphoid process 20 of patient 12. In the example of FIG. 1, lead 16 is implanted at least partially underneath sternum 22 of patient 12. At a location near xiphoid process 20 lead 16 may bend or turn upward and extend superiorly in a substernal position, e.g., within the anterior mediastinum. The anterior mediastinum may be bounded laterally by pleurae, posteriorly by the pericardium, and anteriorly by sternum 22. The distal portion 25 of lead 16 may extend along the posterior side of sternum 22 substantially within the loose connective tissue and/or substernal musculature of anterior mediastinum. A lead implanted such that the distal portion 25 is substantially within anterior mediastinum, or within a pleural cavity or more generally within the thoracic cavity, may be referred to as a “substernal lead.”

In the example illustrated in FIG. 1, lead 16 is located substantially centered under sternum 22. In other instances, however, lead 16 may be implanted such that it is offset laterally from the center of sternum 22. In some instances, lead 16 may extend laterally such that distal portion 25 of lead 16 is underneath/below the ribcage 32 in addition to or instead of sternum 22. In other examples, the distal portion 25 of lead 16 may be implanted in other extra-cardiovascular, intra-thoracic locations, including the pleural cavity or around the perimeter of and adjacent or within the pericardium 38 of heart 8. In various examples, electrodes for sensing cardiac electrical signals can be carried by a lead that may be advanced to a supra-diaphragmatic position, which may be within the thoracic cavity or outside the thorax in various examples.

For instance, lead 16 may bend or turn near xiphoid process 20 to extend superiorly, subcutaneously or submuscularly, over the ribcage and/or sternum, substantially parallel to sternum 22, offset to the right or left of sternum 22, angled laterally from sternum 22 toward the left or the right, or the like. Alternatively, lead 16 may be placed along other subcutaneous or submuscular paths. The path of lead 16 may depend on the location of ICD 14, the arrangement and position of electrodes carried by the lead body 18, and/or other factors. The techniques disclosed herein are not limited to a particular path of lead 16 or final locations of electrodes 24, 26, 28 and 30 for sensing cardiac electrical signals and delivering cardiac pacing therapies and/or CV/DF shocks.

Electrical conductors (not illustrated) extend through one or more lumens of the elongated lead body 18 of lead 16 from the lead connector at the proximal lead end 27 to electrodes 24, 26, 28, and 30 located along the distal portion 25 of the lead body 18. The elongated electrical conductors contained within the lead body 18, which may be separate respective insulated conductors within the lead body 18, are each electrically coupled with respective defibrillation electrodes 24 and 26 and pace/sense electrodes 28 and 30. The respective conductors electrically couple the electrodes 24, 26, 28, and 30 to circuitry, such as a therapy delivery circuit and/or one or more cardiac electrical signal sensing circuits, of ICD 14 via connections in the connector assembly 17, including associated electrical feedthroughs crossing housing 15. The electrical conductors transmit therapy from a therapy delivery circuit within ICD 14 to one or more of defibrillation electrodes 24 and 26 and/or pace/sense electrodes 28 and 30 and transmit cardiac electrical signals sensed from the patient's heart 8 from one or more of defibrillation electrodes 24 and 26 and/or pace/sense electrodes 28 and 30 to the sensing circuit within ICD 14.

The lead body 18 of lead 16 may be formed from a non-conductive material, including silicone, polyurethane, fluoropolymers, mixtures thereof, and/or other appropriate materials, and shaped to form one or more lumens within which the one or more conductors extend. Lead body 18 may be tubular or cylindrical in shape. In other examples, the distal portion 25 (or all of) the elongated lead body 18 may have a flat, ribbon or paddle shape. Lead body 18 may be formed having a preformed distal portion 25 that is generally straight, curving, bending, serpentine, undulating or zigzagging.

In the example shown, lead body 18 includes a curving distal portion 25 having two “C” shaped curves, which together may resemble the Greek letter epsilon, “ε.” Defibrillation electrodes 24 and 26 are each carried by one of the two respective C-shaped portions of the lead body distal portion 25. The two C-shaped curves are seen to extend or curve in the same direction away from a central axis of lead body 18, along which pace/sense electrodes 28 and 30 are positioned. Pace/sense electrodes 28 and 30 may, in some instances, be approximately aligned with the central axis of the straight, proximal portion of lead body 18 such that mid-points of defibrillation electrodes 24 and 26 are laterally offset from pace/sense electrodes 28 and 30. Other examples of leads including one or more defibrillation electrodes and one or more pacing and sensing electrodes may include a curving, serpentine, undulating or zig-zagging distal portion of the lead body 18. The techniques disclosed herein are not limited to any particular lead body design, however. In other examples, lead body 18 is a flexible elongated lead body without any pre-formed shape, bends or curves.

ICD 14 analyzes the cardiac electrical signals received from one or more sensing electrode vectors to monitor for abnormal rhythms, such as bradycardia, ventricular tachycardia (VT) or ventricular fibrillation (VF). ICD 14 may analyze the rate of sensed cardiac events and the morphology of the cardiac electrical signals to monitor for tachyarrhythmia in accordance with any of a number of tachyarrhythmia detection techniques. ICD 14 may generate and deliver electrical stimulation therapy in response to detecting a tachyarrhythmia (e.g., VT or VF) using a therapy delivery electrode vector which may be selected from any of the available electrodes 24, 26, 28 30 and/or housing 15. ICD 14 may deliver anti-tachycardia pacing ATP in response to VT detection, particularly HS VT, and in some cases may deliver ATP prior to a CV/DF shock or during high voltage capacitor charging in an attempt to avert the need for delivering a CV/DF shock. If ATP does not successfully terminate VT or when VF is detected or an HU VT is detected based on EEG signal analysis in combination with cardiac electrical signal analysis, ICD 14 may deliver one or more CV/DF shocks via one or both of defibrillation electrodes 24 and 26 and/or housing 15. ICD 14 may deliver the CV/DF shocks using electrodes 24 and 26 individually or together as a cathode (or anode) and with the housing 15 as an anode (or cathode). As described below, control circuitry of ICD 14 may be configured to select, deliver or withhold an electrical stimulation therapy based on a determination of whether a detected tachyarrhythmia is HU or HS according to an analysis of electrical brain activity that may be sensed by EEG sensor 40. ICD 14 may generate and deliver other types of electrical stimulation pulses such as post-shock pacing pulses, asystole pacing pulses, or bradycardia pacing pulses using a pacing electrode vector that includes one or more of the electrodes 24, 26, 28, and 30 and the housing 15 of ICD 14. In some instances, ICD 14 may detect a slow heart rate that is determined to be HU and deliver bradycardia pacing pulses which may be delivered at a pacing rate that is faster than a programmed lower rate to promote improved blood flow to the brain and hemodynamic stability.

ICD 14 is shown implanted subcutaneously on the left side of patient 12 along the ribcage 32. ICD 14 may, in some instances, be implanted between the left posterior axillary line and the left anterior axillary line of patient 12. ICD 14 may, however, be implanted at other subcutaneous or submuscular locations in patient 12. For example, ICD 14 may be implanted in a subcutaneous pocket in the pectoral region. In this case, lead 16 may extend subcutaneously or submuscularly from ICD 14 toward the manubrium of sternum 22 and bend or turn and extend inferiorly from the manubrium to the desired location subcutaneously, submuscularly, substernally or within ribcage 32. In yet another example, ICD 14 may be placed abdominally. ICD 14 can be communicatively coupled to an external device 50, for example via a wireless link 60, and to EEG sensor 40 and/or cardiac monitor 44 as further described below.

Cardiac monitor 44 may be implanted subcutaneously or submuscularly for sensing cardiac electrical signals via electrodes 46. In FIG. 1, cardiac monitor 44 is implanted inferior to the clavicle and may be implanted in a pectoral, suprasternal, or other thoracic location that enables sensing of cardiac electrical signals via electrodes 46. Cardiac monitor 44 includes a housing 45 enclosing sensing circuitry, communication circuitry, memory, and processing circuitry for analyzing sensed cardiac electrical signals and controlling cardiac monitor functions. Housing 45 may be formed of the types of materials listed above in reference to ICD housing 15. One of electrodes 46a may be carried on a header 47 coupled to housing 45 and electrically coupled to internal device circuitry via an electrical feedthrough extending through the header 47 into the hermetically sealed housing 45. The other electrode 46b may be a conductive portion (or all of) an outer surface of housing 45 and is electrically isolated from electrode 46a. The electrodes 46a and 46b may be formed from the example electrode materials listed above and form a sensing electrode pair for receiving a cardiac electrical signal, e.g., ECG signal, that may be analyzed for detecting a fast heart rate and for detecting and discriminating between different type of arrhythmias. Cardiac monitor 44 may be implemented as an insertable cardiac signal monitor configured to store cardiac signal episodes and detect various cardiac arrhythmias and may generally correspond to the insertable Reveal LINQ™ Heart Monitor available from Medtronic, Inc., Minneapolis, MN, USA, adapted to function in IMD system 10 according to the techniques disclosed herein. Cardiac monitor 44 can be configured to communicate with external device 50, for example, by establishing a wireless link 64.

It is contemplated that cardiac monitor 44 may include neurostimulation capabilities in some examples. Cardiac monitor 44 may implanted in a location for delivering electrical stimulation pulses via electrodes 46 to the patient's nervous system, e.g., to the spinal cord, vagus nerve or other nervous system tissue, for altering the patient's autonomic tone in response to detecting a HU cardiac rhythm in some examples. Electrical neurostimulation may be delivered to increase sympathetic activity to promote or restore hemodynamic stability.

EEG sensor 40 may be a relatively small device, and may be placed (e.g., inserted) under or over the skin in a cranial location or along the patient's neck or base of the skull in various examples. Other target sites at which EEG sensor 40 may be positioned include other positions on the head, such as over the temporal bone. As described in more detail below, EEG sensor 40 may sense electrical brain activity, e.g., one or more EEG signals, corresponding to one or more regions of the patient's brain. EEG sensor 40 can be configured to communicate with external device 50, for example by establishing a wireless link 62.

As described below, EEG sensor 40 can be configured to detect changes in electrical brain activity associated with brain ischemia, which can be indicative of an HU tachyarrhythmia. Accordingly, EEG sensor 40 can include a plurality of electrodes for sensing electrical activity of the patient's brain. In the example shown, EEG sensor 40 includes two electrodes 42a and 42b (collectively electrodes 42) for sensing electrical brain activity but may include more than two electrodes in other examples. In various embodiments, the number and configuration of electrodes 42 can vary. For example, the EEG sensor 40 can include at least 2, at least 3, at least 4, at least 5, or more electrodes in an electrode array. In some embodiments, EEG sensor 40 includes fewer than 6, fewer than 5, fewer than 4, or fewer than 3 electrodes.

The electrodes 42 can be any suitable conductive material or materials, e.g., any of those listed above with regard to electrodes carried by ICD lead 16, to enable EEG sensor 40 to receive electrical signals via electrodes 42. In some examples, the EEG sensor 40 can be configured to analyze data from the electrodes 42 to extract both electrical brain activity data (e.g., EEG signals) and electrical cardiac activity data (e.g., ECG signals).

The electrical brain activity data may be evaluated to provide a determination of brain ischemia while the electrical cardiac activity data may be evaluated to detect a fast heart rate that may be identified as an HU or HS cardiac rhythm based on an analysis of the electrical brain activity data.

In some examples, EEG sensor 40 is configured to analyze data from the electrodes 42 to extract brain activity data and to discard or reduce any contribution from heart or muscle activity. For example, filtering, blind source separation or other techniques may be performed by EEG sensor 40 to remove cardiac, skeletal muscle or other electrical signals from the EEG signal prior to determining EEG signal features or changes that are indicative of brain ischemia. EEG sensor 40 may optionally include other sensors, such as an accelerometer, gyroscope, pulse oximeter, temperature sensor, or any other sensors for monitoring physiological conditions of the patient.

Housing 41 may include a header 43 hermetically sealed to the housing case or can that defines an enclosed inner cavity containing the internal circuitry of EEG sensor 40. Housing 41 is shown as generally rectangular or prismatic in shape in FIG. 1. EEG sensor housing 41 may be generally cylindrical, spherical, angular or other shapes to accommodate a desired number of electrodes and electrode sensing vectors and to accommodate a desired implant location. Electrodes 42 may all be housing-based electrodes. For example, one or more electrodes 42a can be carried by the header 43 hermetically sealed to EEG sensor housing 41. Electrode 42a can be electrically coupled to sensing circuitry within housing 41 via an electrical feedthrough. One or more electrode 42b may be carried on the housing 41, e.g., as a portion of the outer surface of the electrically conductive material forming housing 41, such as any of the example housing materials listed above. Portions of housing 41 may be electrically insulated, e.g., by an insulative coating, to define one or more housing-based electrodes. In some examples, EEG sensor header 43 may be configured to receive a removable lead or be fixedly coupled to a non-removable lead that extends away from housing 41 to carry one or more electrodes for use in sensing electrical signals for monitoring electrical brain activity (and in some instances electrical cardiac activity). In such configurations, a portion of the plurality of electrodes 42 used for sensing electrical brain activity by EEG sensor 40 can be positioned at locations spaced apart from the housing 41.

EEG sensor 40 may be configured as a monitoring only device and implanted at a location for sensing electrical brain activity and, in some examples, electrical cardiac activity. However, it is contemplated that in some examples, EEG sensor 40 includes neurostimulation capabilities. EEG sensor 40 may be positioned for delivering electrical stimulation pulses via electrodes 42 to the patient's nervous system, e.g., to the brain, spinal cord, vagus nerve or other nervous system tissue, for altering the autonomic tone in some examples. In some examples, electrical stimulation may be delivered to increase sympathetic activity to promote or restore hemodynamic stability.

Two or more co-implanted devices in medical device system 10 may be configured to communicate wirelessly, e.g., as represented by wireless communication links 72, 74, and 76, to coordinate the detection of and response to HU and HS cardiac rhythms. ICD 14, EEG sensor 40 and/or cardiac monitor 44 may communicate via radio frequency communication, tissue conductance communication or other communication methods. As described below, an implanted medical device, e.g., cardiac monitor 44 or ICD 14, may detect a concerning heart rate and transmit a request to EEG sensor 40 for a determination of a stage of brain ischemia. EEG sensor 40 may analyze sensed electrical brain activity and transmit an indication of the stage of brain ischemia using techniques described below. The requesting device may receive the transmitted brain ischemia indication for determining whether the concerning heart rate is HU or HS. When a HU cardiac rhythm is detected, the medical device system 10 may respond by transmitting an alert or notification to an external device or communication network to alert medical responders or caregivers. When an implanted medical device of system 10 is capable of delivering a therapy, e.g., ICD 14, a therapy may be delivered or withheld based on the determination of whether the cardiac rhythm is HU or HS.

An external device 50 is shown in telemetric communication with ICD 14, EEG sensor 40, and cardiac monitor 44 by way of wireless communication link 60, wireless communication link 62, and wireless communication link 64, respectively. External device 50 may include a processor 52, memory 53, display unit 54, user interface 56 and telemetry unit 58. Processor 52 controls external device operations and may be configured to process data and signals received from ICD 14, EEG sensor 40 and cardiac monitor 44.

External device 50 may be used to program sensing control parameters, cardiac rhythm detection parameters and therapy delivery control parameters used by ICD 14. External device 50 may be embodied as a programmer used in a hospital, clinic or physician's office to retrieve data from ICD 14, EEG sensor 40 and cardiac monitor 44 and to program operating parameters and algorithms in each of ICD 14, EEG sensor 40 and cardiac monitor 44 used by the respective device for sensing physiological signals and, at least in the case of ICD 14, delivering therapies. External device 50 may alternatively be embodied as a home monitor or handheld device, which may be a tablet, cell phone or other personal device. While a single external device 50 is shown in FIG. 1, it is to be understood that each of ICD 14, EEG sensor 40 and cardiac monitor 44 may be configured to communicate with one or more external devices, such as a system analyzer, programmer, computer, home monitor, communication relay device, cell phone, tablet, or other personal device, any of which may be further in communication with a remote patient monitoring network or database, e.g., the CARELINK™ Remote Monitoring Network, available from Medtronic, Inc., Minneapolis MN, USA. An example IMD programmer that may be configured to communicate with IMDs implementing the techniques disclosed herein is the CARELINK™ Programmer, commercially available from Medtronic, Inc., Minneapolis, Minnesota, USA.

Processor 52 executes instructions stored in memory 53. Processor 52 may include any one or more of a microprocessor, a controller, a digital signal processor (DSP), an application specific integrated circuit (ASIC), a field-programmable gate array (FPGA), or equivalent discrete or analog logic circuitry. In some examples, processor 52 may include multiple components, such as any combination of one or more microprocessors, one or more controllers, one or more DSPs, one or more ASICs, or one or more FPGAs, as well as other discrete or integrated logic circuitry. The functions attributed to processor 52 herein may be embodied as software, firmware, hardware or any combination thereof.

Display 54, which may include a graphical user interface, displays data and other information to a user for reviewing IMD (e.g., ICD 14, EEG sensor 40 or cardiac monitor 44) operation and programmed parameters as well as cardiac electrical signals and/or EEG signals retrieved from the IMDs.

Memory 53 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media. Memory 53 may be configured to store sensing and/or therapy delivery control parameters and associated programmable settings corresponding to a given IMD.

User interface 56 may include a mouse, touch screen, keypad or the like to enable a user to interact with external device 50 to initiate a telemetry session with an IMD implanted in patient 12 for retrieving data from and/or transmitting data to the IMD, including programmable parameters for controlling tachyarrhythmia detection, determination of an HU tachyarrhythmia, and/or therapy delivery. A clinician may use user interface 56 to send and receive commands to an IMD implanted in patient 12 via external device 50. Typically, user interface 56 includes one or more input devices and one or more output devices, including display unit 54. The input devices of user interface 56 may include a communication device such as a network interface, keyboard, pointing device, voice responsive system, video camera, biometric detection/response system, button, sensor, mobile device, control pad, microphone, presence-sensitive screen, touch-sensitive screen (which may be included in display unit 54), network, or any other type of device for detecting input from a human or machine.

The one or more output devices of user interface 56 may include a communication unit such as a network interface, display, sound card, video graphics adapter card, speaker, presence-sensitive screen, one or more USB interfaces, video and/or audio output interfaces, or any other type of device capable of generating tactile, audio, video, or other output. Display unit 54 may function as an input and/or output device using technologies including liquid crystal displays (LCD), quantum dot display, dot matrix displays, light emitting diode (LED) displays, organic light-emitting diode (OLED) displays, cathode ray tube (CRT) displays, e-ink, or monochrome, color, or any other type of display capable of generating tactile, audio, and/or visual output. In other examples, user interface 56 may produce an output to a user in another fashion, such as via a sound card, video graphics adapter card, speaker, presence-sensitive screen, touch-sensitive screen, one or more USB interfaces, video and/or audio output interfaces, or any other type of device capable of generating tactile, audio, video, or other output. In some examples, display unit 54 is a presence-sensitive display that may serve as a user interface device that operates both as one or more input devices and one or more output devices.

Telemetry unit 58 includes a transceiver and antenna configured for bidirectional communication with a telemetry circuit included in an IMD implanted in patient 12 and is configured to operate in conjunction with processor 52 for sending and receiving data relating to IMD functions via a communication link, e.g., any one of the respective communication links 60, 62 or 64. The communication link may be established between an IMD and external device 50 using a radio frequency (RF) link such as BLUETOOTH®, Wi-Fi, or Medical Implant Communication Service (MICS) or other RF or communication frequency bandwidth or communication protocols. As shown in FIG. 1, the individual medical devices of system 10, which may include ICD 14, EEG sensor 40, cardiac monitor 44 and/or other example implantable, partially implantable or wearable medical devices listed herein, may be in communication directly with each other (e.g., as shown by communication links 72, 74 and 76) and/or indirectly with each other via external device 50. For example, at least one of ICD 14, EEG sensor 40, or cardiac monitor 44 may communicate with external device 50 and function as a relay device for transmitting and receiving data from one or more other co-implanted devices. Data stored or acquired by ICD 14, EEG sensor 40 and/or cardiac monitor 44, including cardiac electrical signals or associated data derived therefrom and/or EEG signals or associated data derived therefrom, results of device diagnostics and histories of detected cardiac rhythm episodes, histories of detected brain ischemia, and any delivered therapies may be retrieved from ICD 14, EEG sensor 40 and/or cardiac monitor 44 by external device 50 following an interrogation command. In some examples, the IMD system 10 is configured to detect a HU cardiac rhythm and generate a response by transmitting an alert signal or notification to external device 50 or another external device. In some examples, the alert signal may be a phone call to emergency medical services (EMS). IMD system 10 may be configured to place an emergency phone call via a communication network to alert EMS, or other medical responders of a detected HU cardiac rhythm.

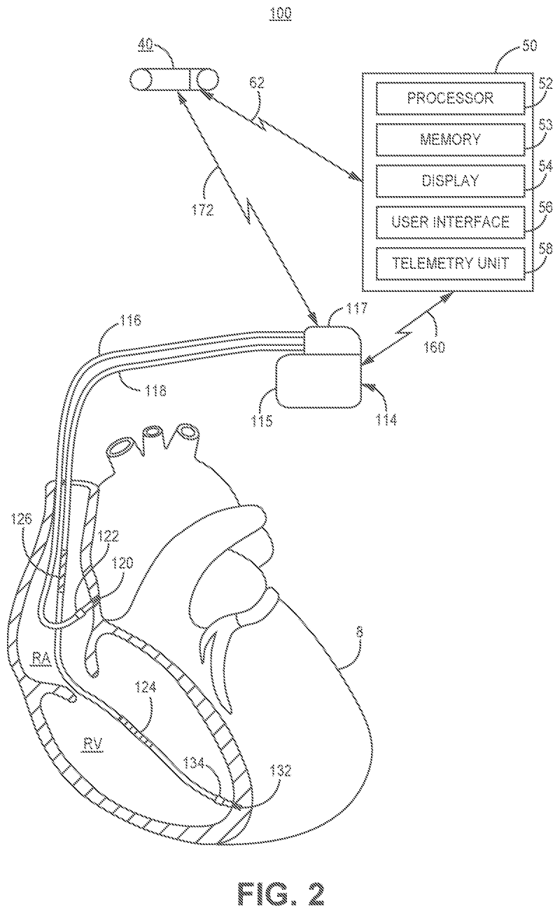

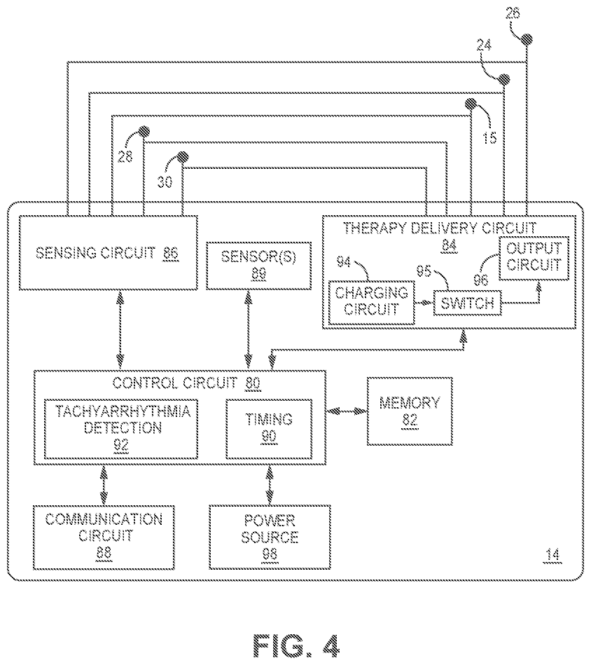

FIG. 2 is a conceptual diagram of EEG sensor 40 and an ICD 114 that may be co-implanted in a medical device system 100 configured to sense at least one cardiac electrical signal and at least one EEG signal for detecting HU and HS cardiac rhythms according to another example. In this example, ICD 114 is shown connected to transvenous, therapy delivery and sensing leads 116 and 118. ICD 14 may be a dual chamber cardiac pacemaker including high voltage CV/DF capabilities. Accordingly, ICD 14 is connected to transvenous leads 116 and 118 in communication with the right atrium (RA) and right ventricle (RV), respectively, of heart 8. ICD 114 includes a housing 115 enclosing circuitry, such as a processing circuitry, telemetry circuitry, sensing circuitry and therapy delivery circuitry, e.g., as generally described below in conjunction with FIG. 4. ICD 114 includes connector assembly 117 having connector bores for receiving proximal connectors of RA lead 116 and RV lead 118 and providing electrical connection between electrodes carried by leads 116 and 118 and internal ICD circuitry.

RA lead 116 may carry a distal tip electrode 120 and ring electrode 122 for sensing atrial electrical signals and producing an atrial intra-cardiac electrogram (EGM) signal. RA electrodes 120 and 122 may be used for delivering RA pacing pulses. RV lead 118 may carry pacing and sensing electrodes 132 and 134 for sensing a ventricular electrical signal and producing an RV EGM signal. RV electrodes 132 and 134 may be used to deliver RV pacing pulses. RV lead 118 may also carry an RV defibrillation electrode 124 and a superior vena cava (SVC) defibrillation electrode 126. Defibrillation electrodes 124 and 126 are shown as coil electrodes spaced apart proximally from the distal pacing and sensing electrodes 132 and 134. While RA lead 116 and RV lead 118 are both shown advanced within a respective heart chamber, in some examples, a transvenous lead coupled to ICD 114 may be advanced to position electrodes within a venous location that is outside the heart 8, such as any of the transvenous, extra-cardiac locations listed above.

ICD 114 may be configured to provide dual chamber sensing and pacing therapies as well as high voltage CV/DF shock therapies in response to detecting VT or VF. In other examples, ICD 114 may be configured to provide multi-chamber sensing and pacing therapies, including cardiac resynchronization therapy (CRT), in which case a coronary sinus lead may be advanced along a cardiac vein to position electrodes for sensing and pacing the left ventricle of heart 8. In still other examples, ICD 114 may be a single chamber device coupled to a single lead, e.g., lead 116 or lead 118, for sensing cardiac electrical signals and delivering electrical stimulation therapies. For instance, ICD 114 may be a single chamber device coupled to RV lead 118 for sensing ventricular EGM signals, detecting and discriminating cardiac tachyarrhythmias, and delivering ventricular pacing, ATP and CV/DF shocks. While RV lead 118 is shown with tip electrode 132 positioned in the RV apex, the location of the leads and electrodes shown are illustrative in nature and not intended to be limiting. The electrodes may be positioned at other locations. For example, RV lead tip electrode 132 may be positioned in the inter-ventricular septum and may be positioned proximate to the His-Purkinje conduction system for delivering conduction system pacing, e.g., in the area of the His bundle, left bundle branch or the right bundle branch.

ICD 114 may be configured to sense at least one cardiac electrical signal for detecting a concerning heart rate. The cardiac electrical signal(s) may be processed and analyzed according to a tachyarrhythmia detection algorithm for detecting a tachyarrhythmia and determining HU tachyarrhythmia or HS tachyarrhythmia in combination with data or information from EEG sensor 40 according to the techniques disclosed herein. ICD 114 and EEG sensor 40 may be configured to communicate wirelessly, as shown by communication link 172, for cooperatively detecting HU and HS cardiac rhythms. Each of EEG sensor 40 and ICD 114 may be capable of bidirectional wireless communication with external device 50, via respective communication links 62 and 160, for transmitting signals or data relating to cardiac rhythms, brain ischemia and for transmitting an alert signal or notification, which may include making an EMS call, and/or calling, texting or emailing a family member or other caregiver or first responder, in response to detecting an HU cardiac rhythm in some examples.

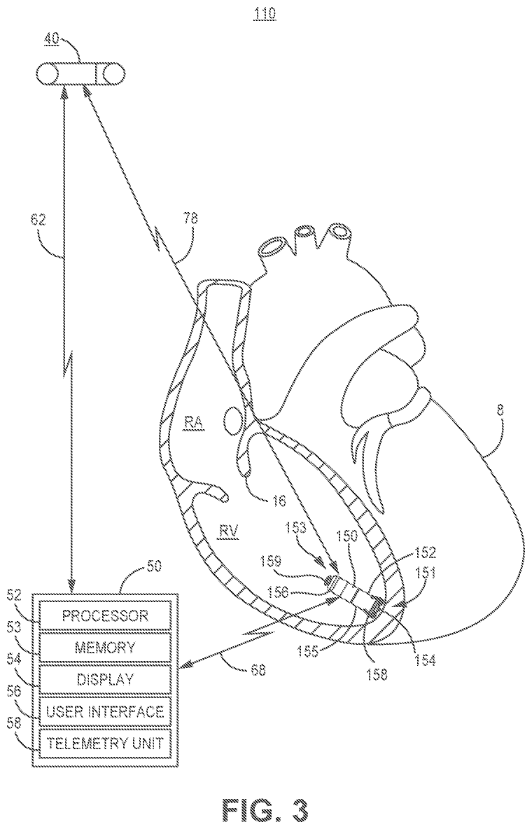

FIG. 3 is a conceptual diagram of a medical device system 110 including EEG sensor 40 and a leadless pacemaker 150. Leadless pacemaker 150 may be configured to sense a cardiac electrical signal for detecting a concerning heart rate and communicate wirelessly with EEG sensor 40 (as shown by communication link 78) for cooperatively detecting HU and HS cardiac rhythms. As shown in FIG. 3, pacemaker 150 may be further configured for bidirectional communication with an external device 50 via a communication link 68.

In some examples, leadless pacemaker 150 may be configured to deliver cardiac electrical stimulation therapy, which may be selected in response to determining whether a concerning heart rate is HU or HS. When a slow heart rate is detected and determined to be HU, leadless pacemaker 150 may deliver bradycardia pacing pulses at a rate that is faster than the sensed intrinsic rate. Leadless pacemaker 150 may be configured to deliver ATP when a tachyarrhythmia is detected or when a fast heart rate is determined to be HU. Leadless pacemaker 150 may deliver ATP based on whether the detected tachyarrhythmia is HS or HU. Leadless pacemaker 150 may deliver ATP in response to detecting a HS tachyarrhythmia and, if the detected tachyarrhythmia is determined to become HU, leadless pacemaker 150 may continue delivering ATP. The medical device system 110 may transmit an alert or make an EMS call in response to detecting an HU cardiac rhythm. In some examples, leadless pacemaker 150 may detect an HU cardiac rhythm cooperatively with EEG sensor 40 for issuing an alert or EMS phone call without necessarily being configured to deliver ATP therapy in response to an HU cardiac rhythm.

While an ICD is not shown in the example of FIG. 3, it is to be understood that leadless pacemaker 150 may be co-implanted with EEG sensor 40 and an ICD, e.g., ICD 14 shown in FIG. 1. Leadless pacemaker 150 may be configured to deliver ATP when an ICD is co-implanted in the patient so that if a tachyarrhythmia is not terminated by ATP delivered by pacemaker 150, the co-implanted ICD may deliver a CV/DF shock to treat and terminate the tachyarrhythmia. For instance, pacemaker 150 may deliver ATP when the detected tachyarrhythmia is determined to be HS and withhold ATP when the detected tachyarrhythmia is determined to be HU if an ICD is co-implanted for delivering a CV/DF shock for terminating the HU tachyarrhythmia.

In still other examples, pacemaker 150 may be co-implanted with cardiac monitor 44 (shown in FIG. 1) and EEG sensor 40. Cardiac monitor 44 may be configured to monitor for a concerning heart rate and/or perform tachyarrhythmia detection algorithms and, cooperatively with EEG sensor 40, determine when a cardiac rhythm is HU. Cardiac monitor 44 may transmit a communication signal to pacemaker 150 for triggering delivery of a therapy, e.g., bradycardia pacing or ATP, by pacemaker 150 based on whether the cardiac rhythm is HU or HS. When an ICD is not present, pacemaker 150 may be triggered to deliver ATP when a detected tachyarrhythmia is HU in an attempt to restore hemodynamic stability. When an ICD is also co-implanted with pacemaker 150, pacemaker 150 may be triggered to deliver ATP as long as the detected tachyarrhythmia is HS. When an HU tachyarrhythmia is detected and an ICD is co-implanted, pacemaker 150 may be inhibited from delivering ATP, and the ICD may deliver a CV/DF shock to terminate the HU tachyarrhythmia.

Pacemaker 150 is shown positioned in the RV, along an endocardial wall, e.g., near the RV apex, though other locations are possible such as along the interventricular septum. The techniques disclosed herein are not limited to the pacemaker location shown in the example of FIG. 3. For example, pacemaker 150 may be positioned in the RA and configured to sense cardiac electrical signals and deliver atrial and/or ventricular pacing from a right atrial location. Pacemaker 150 may be coupled to a lead in some examples for carrying one or more electrodes. Pacemaker 150 could be co-implanted with a second intracardiac pacemaker in a dual chamber pacing system in some examples.

Pacemaker 150 is capable of producing electrical stimulation pulses, e.g., pacing pulses, delivered to heart 8 via electrodes 154 and 156 on the outer housing 152 of the pacemaker. In the location shown, pacemaker 150 is configured to deliver RV pacing pulses and sense an RV cardiac electrical signal using housing based electrodes 154 and 156 for producing an EGM signal. The EGM signal may be sensed using the housing based electrodes 154 and 156 that are also used to deliver pacing pulses to the heart 8.

Electrode 154 is shown as a tip electrode positioned on distal end 151 of pacemaker 150. Electrode 156 is shown as a ring electrode circumscribing a lateral sidewall 155 of housing 152, for example adjacent proximal end 153 of housing 152. Distal end 151 is referred to as “distal” in that it is expected to be the leading end as pacemaker 150 is advanced through a delivery tool, such as a catheter, and placed against a targeted pacing site. Housing 152 may be generally cylindrical in shape to facilitate advancement via a transvenous pathway to an implant site via a catheter or other delivery tool. In other examples, housing 152 may be generally oval or prismatic in shape. Housing 152 can include a longitudinal sidewall 155 extending from housing distal end 151 to housing proximal end 153 to define an interior cavity for housing pacemaker electronics.

Electrodes 154 and 156 can be used as a cathode and anode pair for bipolar cardiac pacing and sensing. Electrodes 154 and 156 may be positioned at locations along pacemaker 150 other than the locations shown. In other examples, pacemaker 150 may include two or more ring electrodes, two or more tip electrodes, and/or other types of electrodes, e.g., a button electrode, a segmented electrode, a helical electrode, a fishhook electrode etc., exposed along pacemaker housing 152 for delivering electrical stimulation to heart 8 and sensing cardiac electrical signals. In some examples, tip electrode 154 is provided as a tissue piercing electrode. Tip electrode 154 may be configured as a tissue piercing electrode for delivering cardiac pacing pulses, including ATP pulses, to a portion of the His-Purkinje conduction system, such as in the area of the His bundle, left or right bundle branch or Purkinje fibers. Electrodes 154 and 156 may be, without limitation, titanium, platinum, iridium or alloys thereof and may include a low polarizing coating, such as titanium nitride, iridium oxide, ruthenium oxide, platinum black, among others.

Housing 152 is formed from a biocompatible material, such as a stainless steel or titanium alloy. In some examples, the housing 152 may include an insulating coating. Examples of insulating coatings include parylene, urethane, PEEK, or polyimide, among others. The entirety of the housing 152 may be insulated, but only electrodes 154 and 156 uninsulated. Electrode 154 may serve as a cathode electrode and be coupled to internal circuitry, e.g., a pacing pulse generator and cardiac electrical signal sensing circuitry, enclosed by housing 152 via an electrical feedthrough crossing housing 152. Electrode 156 may be formed as a conductive portion of housing 152 defining a ring electrode that is electrically isolated from the other portions of the housing 152 as generally shown in FIG. 3. In other examples, the entire periphery of the housing 152 may function as an electrode that is electrically isolated from tip electrode 154, instead of providing a localized ring electrode such as anode electrode 156. Electrode 156 formed along an electrically conductive portion of housing 152 serves as a return anode during pacing and sensing.

Pacemaker 150 may include a set of fixation tines 158 to secure pacemaker 150 to cardiac tissue, e.g., by actively engaging with the ventricular endocardium and/or interacting with the ventricular trabeculae. In a RA location, fixation tines 158 may be inserted in the atrial endocardium to anchor pacemaker 150 at an implant site. Fixation tines 158 are configured to anchor pacemaker 150 to position electrode 154 in operative proximity to a targeted tissue for delivering therapeutic electrical stimulation pulses. Numerous types of active and/or passive fixation members may be employed for anchoring or stabilizing pacemaker 150 at an implant position.

Pacemaker 150 may optionally include a delivery tool interface 159. Delivery tool interface 159 may be located at the proximal end 153 of pacemaker 150 and is configured to connect to a delivery device, such as a catheter, used to position pacemaker 150 at an implant location during an implantation procedure, for example within a heart chamber.

FIG. 4 is a conceptual diagram of an IMD configured to sense cardiac electrical signals and deliver cardiac electrical stimulation therapies according to one example. FIG. 4 is described in conjunction with the ICD 14 of FIG. 1, including relatively low voltage cardiac pacing therapy delivery capabilities and relatively higher voltage CV/DF therapy delivery capabilities. It is to be understood, however, that the circuitry and functionality attributed to circuitry described in conjunction with FIG. 4 may be included, in whole or in part, in any of the example IMDs described or listed herein that are configured to sense at least one cardiac electrical signal and detect a concerning heart rate, as such cardiac monitor 44, ICD 114, or pacemaker 150.

The ICD housing 15 is shown schematically as an electrode in FIG. 4 since the housing of ICD 14 may be used as an electrode in a sensing electrode vector for cardiac signal sensing and/or for therapy delivery in some examples. The electronic circuitry enclosed within housing 15 includes software, firmware and hardware that cooperatively monitor cardiac signals, determine when an electrical stimulation therapy is necessary, and deliver therapy as needed according to programmed therapy delivery algorithms and control parameters.

Control circuit 80 communicates, e.g., via a data bus, with therapy delivery circuit 84 and sensing circuit 86 for sensing cardiac event signals, detecting cardiac rhythms, and controlling delivery of cardiac electrical stimulation therapies in response to sensed cardiac event signals. Therapy delivery circuit 84 and sensing circuit 86 are electrically coupled to electrodes 24, 26, 28, 30 (e.g., carried by lead 16 as shown in FIG. 1) and the housing 15, which may function as a common or ground electrode or as an active can electrode for delivering CV/DF shock pulses or cardiac pacing pulses. As described above electrodes 24, 26, 28 and 30 shown in FIG. 4 may be carried by a non-transvenous lead advanced to position electrodes in a substernal, subcutaneous or submuscular, extra-cardiac location (e.g., as shown in FIG. 1). In other examples, electrodes coupled to sensing circuit 86 and/or therapy delivery circuit 84 may be carried by a transvenous lead for positioning electrodes within a blood vessel or an intracardiac location (e.g., electrodes 120, 122, 124, 126, 132, and 134 as shown in FIG. 2). Furthermore, electrodes coupled to the ICD (or a pacemaker such as pacemaker 150) may include multiple housing-based electrodes not carried by a lead in some examples.

ICD 14 as shown in FIG. 4 includes a control circuit 80, memory 82, therapy delivery circuit 84, cardiac electrical signal sensing circuit 86, communication circuit 88 and optionally one or more physiological sensors 89. A power source 98 provides power to the circuitry of ICD 14, including each of the components 80, 82, 84, 86, and 88 as needed. Power source 98 may include one or more energy storage devices, such as one or more rechargeable or non-rechargeable batteries. The connections between power source 98 and each of the other components 80, 82, 84, 86 and 88 are to be understood from the general block diagram of FIG. 4 but are not shown for the sake of clarity. For example, power source 98 may be coupled to one or more charging circuits included in therapy delivery circuit 84 for charging holding capacitors or other charge storage devices included in therapy delivery circuit 84 that are discharged at appropriate times under the control of control circuit 80 for producing electrical pulses according to a therapy protocol. In other examples, power source 98 may serve as a voltage or current source to therapy delivery circuit 84 without requiring a charge storage device. Power source 98 is also coupled to components of cardiac electrical signal sensing circuit 86, such as sense amplifiers, analog-to-digital converters, switching circuitry, etc. as needed.

The circuits shown in FIG. 4 represent functionality included in ICD 14 or another medical device operating according to the techniques disclosed herein and may include any discrete and/or integrated electronic circuit components that implement analog and/or digital circuits capable of producing the functions attributed to ICD 14 herein. Functionality associated with one or more circuits may be performed by separate hardware, firmware or software components, or integrated within common hardware, firmware or software components. For example, cardiac event sensing and determination of sensed cardiac event intervals for detecting a concerning heart rate and/or tachyarrhythmia detection may be performed cooperatively by sensing circuit 86 and control circuit 80 and may include operations implemented in a processor or other signal processing circuitry included in control circuit 80 executing instructions stored in memory 82. Control signals such as blanking and timing intervals associated with cardiac event signal sensing, e.g., R-wave sensing and/or P-wave sensing, may be sent from control circuit 80 to sensing circuit 86 according to programmed sensing control parameter settings.

The various circuits of ICD 14 may include an application specific integrated circuit (ASIC), an electronic circuit, a processor (shared, dedicated, or group) and memory that execute one or more software or firmware programs, a combinational logic circuit, state machine, or other suitable components or combinations of components that provide the described functionality. The particular form of software, hardware and/or firmware employed to implement the functionality disclosed herein will be determined primarily by the particular system architecture employed in the medical device system and by the particular detection and therapy delivery methodologies employed by the medical device system. Providing software, hardware, and/or firmware to accomplish the described functionality in the context of any modern medical device, given the disclosure herein, is within the abilities of one of skill in the art.

Memory 82 may include any volatile, non-volatile, magnetic, or electrical non-transitory computer readable storage media, such as random access memory (RAM), read-only memory (ROM), non-volatile RAM (NVRAM), electrically-erasable programmable ROM (EEPROM), flash memory, or any other memory device. Furthermore, memory 82 may include non-transitory computer readable media storing instructions that, when executed by one or more processing circuits, cause control circuit 80 and/or other ICD components to perform various functions attributed to ICD 14 or those ICD components. The non-transitory computer-readable media storing the instructions may include any of the media listed above.

Cardiac electrical signal sensing circuit 86 (also referred to herein as “sensing circuit” 86) may be selectively coupled to electrodes 28, 30 and/or housing 15 in order to sense electrical activity of the patient's heart. Sensing circuit 86 may additionally be selectively coupled to defibrillation electrodes 24 and/or 26 for use in a sensing electrode vector together or in combination with one or more of electrodes 28, 30 and/or housing 15. Sensing circuit 86 may be enabled to selectively receive cardiac electrical signals from one or more different sensing electrode vectors from the available electrodes 24, 26, 28, 30, and housing 15 in some examples. Sensing circuit 86 may monitor one or more cardiac electrical signals for sensing cardiac event signals attendant to myocardial depolarizations and/or producing digitized cardiac electrical signals passed to control circuit 80 for processing and analysis and/or for further transmission to external device 50 via telemetry circuit 88. For example, sensing circuit 86 may include switching circuitry for selecting which of electrodes 24, 26, 28, 30, and housing 15 are coupled to one or more sensing channels of sensing circuit 86.

Sensing circuit 86 may be configured to amplify, filter, rectify and digitize or otherwise process the cardiac electrical signal received from each selected sensing electrode vector to improve the signal quality for sensing cardiac electrical event signals, such as R-waves attendant to ventricular myocardial depolarization and P-waves attendant to atrial myocardial depolarization. Cardiac event detection circuitry included within sensing circuit 86 may include one or more sense amplifiers, filters, rectifiers, threshold detectors, comparators, analog-to-digital converters (ADCs), timers or other analog or digital components configured to sense cardiac event signals.

Sensing circuit 86 may control the amplitude of an auto-adjusting cardiac event sensing threshold over each cardiac cycle. Sensing circuit 86 may sense a cardiac event signal in response to the received cardiac electrical signal crossing the sensing threshold. Sensing circuit 86 may produce a cardiac sensed event signal, e.g., an atrial sensed event signal in response to a P-wave sensing threshold crossing or a ventricular sensed event signal in response to an R-wave sensing threshold crossing. The cardiac sensed event signals can be passed to control circuit 80 to indicate the timing of a sensing threshold crossing. Various sensing threshold control parameters may be used by sensing circuit 86 to set and adjust the cardiac event sensing threshold during each cardiac cycle. These sensing threshold control parameters may be stored in memory 82 and passed to sensing circuit 86 from control circuit 80 for use by hardware, firmware and/or software of control circuit 80 and/or sensing circuit 86 in controlling the amplitude of the cardiac event sensing threshold according to one or more blanking periods, refractory periods, sense delay times, drop time intervals, decay time intervals, sensitivities or other sensing threshold control parameters.

Control circuit 80 may receive cardiac sensed event signals from sensing circuit 86 for determining sensed event intervals, e.g., RR intervals (RRIs) and/or PP intervals (PPIs), by timing circuit 90. An RRI is the time interval between two consecutively sensed R-waves and may be determined between consecutive ventricular sensed event signals received by control circuit 80 from sensing circuit 86. A PPI is the time interval between two consecutively sensed P-waves and may be determined between consecutive atrial sensed event signals received by control circuit 80 from sensing circuit 86. Depending on programmed therapies, timing circuit 90 may trigger therapy delivery circuit 84 to generate and deliver an electrical stimulation pulse in response to a sensed event signal and/or start a pacing escape interval timer in response to a sensed event signal and restart the escape interval timer in response to the next sensed event signal. The value of the escape interval timer at the time of the next sensed event signal may be buffered in memory 82 as the sensed event interval for the associated sensed event signal. In this way, memory 82 may store a series of cardiac sensed event intervals, e.g., RRIs, for determining a sensed heart rate and detecting a concerning heart rate.

As further described below, when a concerning heart rate is detected, ICD 14 may request an analysis of electrical brain activity by EEG sensor 40 in some examples. ICD 14 may detect a concerning heart rate when RRIs (or PPIs) meet fast heart rate criteria. Additionally or alternatively, ICD 14 may detect a concerning heart rate when RRIs (or PPIs) meet slow heart rate criteria. Criteria for detecting a concerning heart rate may be programmed by a user and may be tailored for an individual patient according to patient need.

For example, when a threshold number of RRIs or a median or average RRI out of a most recent specified number of RRIs is less than a fast rate threshold interval, a fast heart rate may be detected. Detection of a fast heart rate may or may not meet tachyarrhythmia detection requirements, such as VT or VF detection requirements. When a fast heart rate is detected, the medical device system may determine whether the cardiac rhythm is HU or HS. The HU or HS determination may be made before or after a tachyarrhythmia is detected or when a tachyarrhythmia detection is made with a relatively low confidence level or low probability level.

ICD 14 may be configured to detect a slow heart rate when a threshold number of RRIs (or PPIs) or a median or average RRI out of a most recent specified number of RRIs is greater than a slow rate threshold interval. Detection of a slow heart rate may be a heart rate than is slower or faster than a programmed lower pacing rate. Some patients may experience reduced brain perfusion when the heart rate is reduced, but possibly still faster than a programmed lower pacing rate, particularly in combination with a drop in blood pressure. As such, a slow heart rate threshold may be defined for detecting a concerning heart rate by control circuit 80 based on sensed event signals received from sensing circuit 86. The slow heart rate may be determined to be HU or HS based on analysis of the sensed electrical brain activity.

Timing circuit 90 may include various timers and/or counters used to control the timing of therapy delivery by therapy delivery circuit 84. In response to expiration of an escape interval timer without receiving a cardiac sensed event signal, control circuit 80 may control therapy delivery circuit 84 to generate and deliver a pacing pulse. Timing circuit 90 may additionally set time windows such as morphology template windows, morphology analysis windows or perform other timing related functions of ICD 14 including synchronizing CV/DF shocks or other therapies delivered by therapy delivery circuit 84 with sensed cardiac events.

Control circuit 80 may include a tachyarrhythmia detection circuit 92 configured to analyze signals received from sensing circuit 86 for detecting tachyarrhythmia. Tachyarrhythmia detection circuit 92 may detect tachyarrhythmia based on cardiac events sensed by sensing circuit 86 meeting tachyarrhythmia detection criteria, such as a threshold number of sensed cardiac event signals occurring at sensed event intervals falling in a tachyarrhythmia interval range. Tachyarrhythmia detection circuit 92 may be implemented in control circuit 80 as hardware, software and/or firmware that processes and analyzes signals received from sensing circuit 86 for detecting tachyarrhythmia, e.g., supraventricular tachycardia (SVT), VT and/or VF. Tachyarrhythmia detection circuit 92 may include comparators and counters for counting cardiac event intervals, e.g., PPIs and/or RRIs determined by timing circuit 90, that fall into various rate detection zones for determining an atrial rate and/or a ventricular rate or performing other rate- or interval-based assessment of cardiac sensed event signals for detecting and discriminating tachyarrhythmias.

For example, tachyarrhythmia detection circuit 92 may compare the RRIs determined by timing circuit 90 to one or more tachyarrhythmia detection interval zones, such as a tachycardia detection interval zone and a fibrillation detection interval zone. RRIs falling into a detection interval zone are counted by a respective VT interval counter or VF interval counter and in some cases in a combined VT/VF interval counter included in tachyarrhythmia detection circuit 92. The VF detection interval threshold may be set to 300 to 350 milliseconds (ms), as an example. For instance, if the VF detection interval is set to 320 ms, RRIs that are less than 320 ms are counted by the VF interval counter. When VT detection is enabled, the VT detection interval may be programmed to be in the range of 350 to 420 ms, or 400 ms as an example. RRIs that are less than the VT detection interval but greater than or equal to the VF detection interval may be counted by a VT interval counter. In order to detect VT or VF, the respective VT or VF interval counter is required to reach a threshold “number of intervals to detect” or “NID.”

As an example, the NID to detect VT may require that the VT interval counter reaches 18 VT intervals, 24 VT intervals, 32 VT intervals or other selected NID. In some examples, the VT intervals may be required to be consecutive intervals, e.g., 18 out of 18, 24 out of 24, or 32 out of the most recent 32 consecutive RRIs. The NID required to detect VF may be programmed to a threshold number of X VF intervals out of Y consecutive RRIs. For instance, the NID required to detect VF may be 18 VF intervals out of the most recent 24 consecutive RRIs or 30 VF intervals out 40 consecutive RRIs, as examples.

When a VT or VF interval counter reaches an NID, a ventricular tachyarrhythmia may be detected by tachyarrhythmia detection circuit 92. The NID may be programmable and range from as low as 12 to as high as 100, with no limitation intended. VT or VF intervals may be detected consecutively or non-consecutively out of a specified number of most recent RRIs. In some cases, a combined VT/VF interval counter may count both VT and VF intervals and detect a tachyarrhythmia episode based on the fastest intervals detected when a specified NID is reached.

Tachyarrhythmia detection circuit 92 may be configured to perform other signal analysis for determining if other detection criteria are satisfied before detecting VT or VF, such as R-wave morphology criteria and onset criteria. To support additional cardiac signal analyses, sensing circuit 86 may pass a digitized cardiac electrical signal, e.g., an electrocardiogram (ECG) signal when sensed using electrodes outside the heart or an EGM signal when sensed using intracardiac electrodes, to control circuit 80 for morphology analysis performed by tachyarrhythmia detection circuit 92 for detecting and discriminating cardiac rhythms. A cardiac electrical signal from a selected sensing electrode vector may be passed through a filter and amplifier, provided to a multiplexer and thereafter converted to a multi-bit digital signal by an analog-to-digital converter, all included in sensing circuit 86, for storage in memory 82 and/or for real time transmission via telemetry circuit 88. Memory 82 may include one or more circulating buffers to temporarily store digital cardiac electrical signal segments (or episodes) for analysis performed by control circuit 80 and/or by external device processor 52 after transmission via telemetry circuit 88. Control circuit 80 may be a microprocessor-based controller that employs digital signal analysis techniques to characterize the digitized signals stored in memory 82 to recognize and classify the patient's cardiac rhythm employing any of numerous signal processing methodologies for analyzing cardiac electrical signals and cardiac event waveforms, e.g., R-waves.

In some examples, control circuit 80 may establish an R-wave morphology template that is stored in memory 82 and compared to a sensed cardiac electrical signal waveform for determining a morphology matching score. Wavelet transform techniques may be used for determining a morphology matching score in some examples. Morphology matching scores may be determined by tachyarrhythmia detection circuit 92 for discriminating between fast cardiac rhythms, such as an SVT, monomorphic VT, polymorphic VT or VF.

Therapy delivery circuit 84 includes at least one charging circuit 94, including one or more charge storage devices such as one or more high voltage capacitors and/or low voltage capacitors, and switching circuitry 95 that controls when the charge storage device(s) are discharged through an output circuit 96 across a selected pacing electrode vector or CV/DF shock vector. Charging of capacitors to a programmed pulse amplitude and discharging of the capacitors for a programmed pulse width may be performed by therapy delivery circuit 84 according to control signals received from control circuit 80 for delivering cardiac pacing pulses. As described above, timing circuit 90 may include various timers or counters that control when cardiac pacing pulses are delivered. The microprocessor of control circuit 80 may set the amplitude, pulse width, polarity or other characteristics of cardiac pacing pulses, which may be based on programmed values stored in memory 82.

In response to detecting VT or VF, control circuit 80 may schedule a therapy and control therapy delivery circuit 84 to generate and deliver the therapy, such as ATP and/or CV/DF therapy. Therapy may be selected based on a determination of HS tachyarrhythmia or HU tachyarrhythmia using EEG signals sensed by EEG sensor 40 as further described below. Therapy can be generated by initiating charging of high voltage capacitors in charging circuit 94. Charging is controlled by control circuit 80 which monitors the voltage on the high voltage capacitors, which is passed to control circuit 80 via a charging control line. When the voltage reaches a predetermined value set by control circuit 80, a logic signal can be generated on a capacitor full line and passed to therapy delivery circuit 84, terminating charging. A relatively high voltage pacing pulse or a CV/DF pulse can be delivered to the heart, e.g., using electrodes 24, 26 and or housing 15, under the control of the timing circuit 90 by an output circuit 96 of therapy delivery circuit 84 via a control bus. The output circuit may include an output capacitor through which the charged high voltage capacitor is discharged via switching circuitry, e.g., an H-bridge, which determines the electrodes used for delivering a CV/DF shock pulse and the pulse wave shape.

In some examples, the high voltage therapy circuit configured to deliver CV/DF shock pulses can be controlled by control circuit 80 to deliver pacing pulses, e.g., for delivering ATP, post shock pacing pulses or other ventricular pacing pulses. In other examples, therapy delivery circuit 84 may include a low voltage therapy circuit for generating and delivering pacing pulses for a variety of pacing needs. When the medical device system is configured to determine when a slow heart rate is HU, therapy delivery circuit may be controlled by control circuit 80 to deliver bradycardia pacing pulses at a rate faster than the sensed slow heart rate to improve or restore hemodynamic stability.

It is recognized that the methods disclosed herein for processing and analyzing cardiac electrical signals and EEG signals may be implemented in a medical device system that includes a cardiac monitor 44 (FIG. 1) for monitoring cardiac electrical signals by a sensing circuit 86 and a control circuit 80 without necessarily having therapy delivery capabilities (e.g., no therapy delivery circuit 84) or in a medical device system that includes a pacemaker, e.g., pacemaker 150 (FIG. 3), that monitors cardiac electrical signals and delivers cardiac pacing therapies by therapy delivery circuit 84, without high voltage therapy capabilities such as CV/DF shock capabilities.

Sensor(s) 89 may include one or more of an accelerometer, gyroscope, pressure sensor, temperature sensor, oxygen sensor, impedance sensor, or other physiological sensor for sensing a signal responsive to changes in a physiological condition of the patient. For example, sensor(s) 89 may include an accelerometer for sensing patient physical activity and/or patient posture. Physical activity and/or patient posture may be used for detecting patient sleep in some examples. EEG signals during sleep can resemble EEG signals during brain ischemia. As described below, in some examples, when EEG signal analysis indicates reduced brain perfusion and a possible HU cardiac rhythm, the medical device system may determine whether or not the patient is likely to be asleep. If the patient is asleep, a determination of brain ischemia may be indeterminable or low confidence. Sleep may be detected based on a horizonal or reclining posture (e.g., a non-upright posture) and/or a non-resting level of patient physical activity, one or both of which may be determined by control circuit 80 based on an accelerometer signal received from sensors 89. Decreased body temperature and/or respiration rate are other examples of that may be determined from a sensor signal received by control circuit 80 from sensor(s) 89 for use in detecting sleep for use in discriminating changes in electrical brain activity due to brain ischemia versus sleep or other physiological conditions.

In other examples, when brain ischemia is detected based on EEG signal analysis, other physiological signals may be analyzed for confirming or verifying the likelihood of brain ischemia and a HU cardiac rhythm. For example, a decrease in oxygen saturation and/or blood pressure may be detected from a sensor signal received by control circuit 80 from sensors 89. A decrease in oxygen saturation and/or blood pressure may be used by control circuit 80 in confirming brain ischemia detected from electrical brain activity, indicating a HU cardiac rhythm. While sensors 89 are shown included in ICD 14 in FIG. 4, it is to be understood that any of the example sensors listed herein may be included in one or more of the medical devices included in the medical device system performing the methods disclosed herein. For example, any of EEG sensor 40, cardiac monitor 44, ICD 114 and/or pacemaker 150 may include one or more of the sensors listed above for use in detecting sleep that may result in a brain ischemia condition being indeterminate and/or for detecting physiological signal changes that can be used to confirm a brain ischemia detection and corresponding HU cardiac rhythm.

Control parameters utilized by control circuit 80 for sensing cardiac event signals, detecting tachyarrhythmias and controlling therapy delivery may be programmed into memory 82 via communication circuit 88. Communication circuit 88 includes a transceiver and antenna for communicating with external device 50 (shown in FIG. 1) using RF communication such as Bluetooth or other communication protocols as described above. Under the control of control circuit 80, communication circuit 88 may receive downlink telemetry from and send uplink telemetry to external device 50. Communication circuit 88 may transmit sensed cardiac electrical signals (and in some cases sensed cardiac event markers and associated sensed event intervals) to another medical device, e.g., external device 50, for processing and analysis and/or display by external device 50.

Communication circuit 88 may be configured to transmit and receive data using RF telemetry, such as Bluetooth or other wireless RF transmission protocols to a co-implanted IMD. However, in some examples, ICD 14, EEG sensor 40, cardiac monitor 44, ICD 114 and/or pacemaker 150 when co-implanted with one or more other IMDs in a medical device system performing the techniques disclosed herein, may communicate using tissue conduction communication (TCC). Examples of TCC apparatus and methods that may be implemented in an IMD system configured to discriminate between HS and HU cardiac rhythms are generally disclosed in U.S. Pat. No. 9,636,511 (Carney, et al.), U.S. Pat. No. 11,213,684 (Peichel, et al.), U.S. Pat. No. 11,235,162 (Reinke, et al), U.S. Pat. No. 11,045,654 (Peichel, et al.) and U.S. Pat. No. 11,110,279 (Roberts, et al.). For example, upon detecting a concerning heart rate or when tachyarrhythmia detection criteria are met, ICD 14 may transmit a TCC signal via communication circuit 88, e.g., using electrodes 28 and 30 or other electrodes coupled to ICD 14, to request analysis of electrical brain activity by EEG sensor 40 for determining if the cardiac rhythm is HU or HS. Communication circuit 88 may receive an RF signal or a TCC signal transmitted by EEG sensor 40 indicating whether brain ischemia is detected at a level or stage that indicates an HU cardiac rhythm or not.

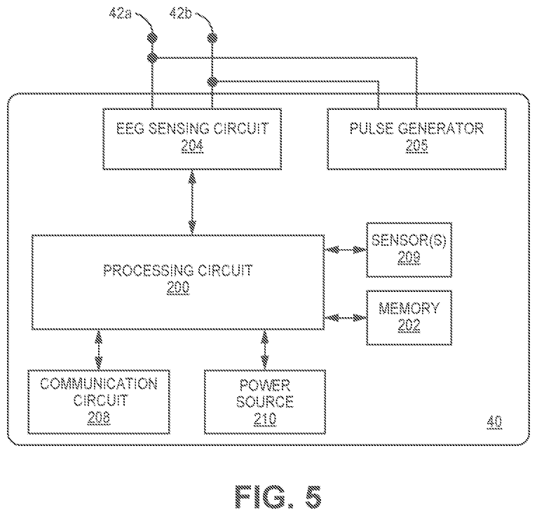

FIG. 5 is a conceptual diagram of electronic circuitry that can be included in EEG sensor 40 according to one example. EEG sensor 40 may include processing circuit 200, memory 202, EEG sensing circuit 204, communication circuit 208, and power source 210. As indicated above, EEG sensor 40 may include one or more sensor(s) 209 for sensing a signal responsive to changes in a physiological condition of a patient for use in detecting brain ischemia. Any of the example sensors listed herein may be included in sensor(s) 209. EEG sensor 40 may optionally include a pulse generator 205 when EEG sensor 40 includes neurostimulation capabilities. As mentioned above, an IMD included in the medical device system performing the techniques disclosed herein may be configured to deliver neurostimulation for altering the patient's autonomic tone in response to detecting a HU cardiac rhythm. Pulse generator 205 may be included in EEG sensor 40 (or cardiac monitor 44 in other examples) for delivering neurostimulation pulses via electrodes 42 in response to a therapy control signal generated to provide a response to a HU cardiac rhythm detection. Power source 210 provides power to the circuitry of EEG sensor 40, including each of the circuits 200, 202, 204, 205, 206 and 208 as needed. Power source 210 may include one or more energy storage devices, such as one or more rechargeable or non-rechargeable batteries.

The functional blocks shown in FIG. 5 represent functionality that may be included in EEG sensor 40 implemented as one or more discrete and/or integrated electronic circuit components that implement analog and/or digital circuits capable of performing the functions attributed to EEG sensor 40 herein. Processing circuit 200 can include one or more central processing units, ASICs, digital signal processing circuitry, or any other suitable electrical components configured to process and analyze signals from EEG sensing circuit 204 and control operation of the EEG sensor 40. Processing circuit 200 may include hardware particularly adapted for artificial intelligence (AI) or machine learning applications, for example, a tensor processing unit (TPU) or other such hardware.

Memory 202 may include any volatile, non-volatile, magnetic, or electrical non-transitory computer readable storage media, such as any of the examples listed above. Furthermore, memory 202 may include non-transitory computer readable media storing instructions that, when executed by processing circuit 200 (and/or other processing circuitry included in EEG sensing circuit 204 and/or communication circuit 208), cause processing circuit 200 to perform various functions attributed to EEG sensor 40. The non-transitory computer readable media storing the instructions may include any of the media listed above.

EEG sensor 40 can be configured to sense electrophysiology signals corresponding to electrical brain activity and analyze the signals to detect changes in electrical brain activity indicative of brain ischemia that may be associated with an HU cardiac rhythm. Electrophysiology signals are received by EEG sensing circuit 204 via electrodes 42. While only two electrodes are shown in FIG. 5, it is contemplated that three or more electrodes may be coupled to EEG sensor 40 for sensing electrophysiology signals for monitoring electrical brain activity, which may include separating or extracting an EEG signal from other electrophysiological signals and/or non-physiological noise.

EEG sensor 40 can be implanted at a location to detect electrical activity corresponding to brain activity (e.g., EEG data) but, depending on the implant location, may receive cardiac electrical signals and/or skeletal muscle electrical signals and/or other electrophysiological signals. EEG sensing circuit 204 may be configured to filter, amplify and digitize an incoming electrophysiological signal for removing electrical noise, cardiac electrical signals, skeletal muscle signals or other non-EEG signals for passing an EEG signal to processing circuit 200. EEG sensing circuit 204 may include one or more filter(s), amplifier(s), analog to digital convertor(s) (ADCs), rectifiers, comparators, or other circuitry for receiving a signal from electrodes 42 and passing an EEG signal to processing circuit 200 representative of electrical brain activity. For instance, EEG sensing circuit 204 can include one or more input protection circuits to filter the electrical signals sensed via electrodes 42 and may include amplifier/filter circuitry to remove DC and high frequency components or any other suitable components.

In some examples, processing circuit 200 may be further configured to process the incoming EEG signal to remove cardiac electrical signals or other non-EEG signal content from the EEG signal, e.g., using blind source separation, or other techniques. Processing circuit 200 can be configured to analyze the EEG signal to determine one or more EEG metrics for detecting brain ischemia. In some examples, as further described below, changes in EEG frequency content and/or amplitude may be detected by processing circuit 200 to identify two or more stages of brain ischemia, which could also be referred to as levels or degrees of brain ischemia. It should be understood, however, that in some examples processing circuit 200 may control communication circuit 208 to transmit an EEG signal episode to another co-implanted device (or external device 50) included in the medical device system for processing and analysis to detect brain ischemia indicative of decreased cerebral blood flow, which may be due to an HU tachyarrhythmia. The detected stage of brain ischemia may be used by one or more co-implanted IMD(s), e.g., ICD 14, cardiac monitor 44, ICD 114 and/or pacemaker 150, for generating a response to an HU cardiac rhythm, which may include selecting a cardiac electrical stimulation therapy response (which may include withholding, delaying and/or delivering a cardiac electrical stimulation therapy) and/or generating an alert to notify the patient, a clinician, a first responder or other caregiver of the HU cardiac rhythm, which may include calling EMS via a communications network.

In some examples, processing circuit 200 may be configured to analyze the sensed signal(s) received from sensing circuit 204 to extract both an EEG signal and a cardiac electrical signal, e.g., an ECG signal. The EEG signal may be analyzed by processing circuit 200 for detecting brain ischemia. The ECG signal may be analyzed by processing circuit 200 for detecting a concerning heart rate (e.g., less than a slow heart rate threshold and/or faster than a fast heart rate threshold) and/or detecting and discriminating tachyarrhythmias, e.g., atrial fibrillation, sinus tachycardia or other SVT, monomorphic VT, polymorphic VT and/or VF. In this way, EEG sensor 40 may perform techniques disclosed herein for detecting HU and HS cardiac rhythms and generating an alert or notification of an HU tachyarrhythmia without requiring another co-implanted IMD. In other examples, EEG sensor 40 may be co-implanted with an IMD, such as ICD 14, ICD 114 or pacemaker 150 that is capable of delivering an electrical stimulation therapy selected based on whether the cardiac rhythm is determined to be HU or HS. For instance, in response to detecting an HU tachyarrhythmia, processing circuit 200 may control communication circuit 208 to transmit a communication signal indicating the detection of brain ischemia or an HU cardiac rhythm to a co-implanted IMD capable of delivering cardiac pacing, ATP, a CV/DF shock, vagal nerve stimulation, spinal cord stimulation or other neurostimulation. When brain ischemia is not detected or is at less than a threshold stage of brain ischemia, communication circuit 208 may transmit a communication signal to a co-implanted IMD indicating the detection of no brain ischemia or an HS cardiac rhythm. The co-implanted IMD may or may not deliver a therapy when a HS cardiac rhythm is detected. For example, a slow heart rate that is HS may not warrant any therapy delivery. However, a fast heart rate that is detected as VT may warrant delivery of ATP to terminate the HS ventricular tachycardia before it becomes HU, for example.

In other examples, processing circuit 200 is configured to analyze signals sensed by electrodes 42 and received from EEG sensing circuit 204 to extract EEG signal data and to discard or reduce any contribution from heart or muscle activity. Processing circuit 200 may be configured to determine one or more EEG metrics that can be transmitted via communication circuit 208 for receipt a co-implanted IMD (or to external device 50). The EEG metrics may be transmitted when brain ischemia is detected or in response to request from a co-implanted IMD that may be detecting a concerning heart rate. The receiving IMD (or external device 50) may determine a stage of brain ischemia based on the EEG metrics and perform an appropriate response such as generating an alert signal (e.g., for calling EMS or notifying a clinician, first responder, family member or other caregiver), delivering, delaying or withholding a therapy, performing continued monitoring of the EEG signal, or other responses as described herein. In other examples, processing circuit 200 determines the EEG metrics and determines a stage of brain ischemia based on the EEG metrics. Communication circuit 208 may transmit an indication of the stage of brain ischemia to a co-implanted IMD (or external device 50) that uses the brain ischemia determination for discriminating between HS and HU cardiac rhythms and selecting an appropriate response.

In some examples communication circuit 208 may include an antenna and RF transceiver for transmitting and receiving data for cooperatively detecting HU cardiac rhythms in combination with one or more co-implanted IMDs (and/or external device). Communication circuit 208 may also be configured to transmit an alert when brain ischemia is detected based on electrical brain activity. Additionally or alternatively, communication circuit 208 may be configured to send and receive TCC signals via electrodes 42. For example, communication circuit 208 may be configured to receive and demodulate a TCC signal received via electrodes 42 that triggers processing circuit 200 to analyze an incoming EEG signal for determining one or more EEG metrics and determining a stage of brain ischemia. Communication circuit 208 may transmit a TCC signal via electrodes 42 to another co-implanted IMD, e.g., ICD 14, cardiac monitor 44, ICD 114, and/or pacemaker 150, to indicate detection of brain ischemia and/or to communicate a stage (or level or degree) of brain ischemia. Communication circuit 208 may include an oscillator generating a carrier signal that is modulated to produce a TCC signal, which may be 10 kHz to 100 kHz in frequency and amplified to an amplitude of 50 to 500 millivolts in some examples. Examples of TCC communication circuitry and techniques that may be implemented in an IMD system performing the methods disclosed herein are generally disclosed in the above-incorporated U.S. Pat. No. 9,636,511 (Carney, et al.), U.S. Pat. No. 11,213,684 (Peichel, et al.), U.S. Pat. No. 11,235,162 (Reinke, et al), U.S. Pat. No. 11,045,654 (Peichel, et al.)and U.S. Pat. No. 11,110,279 (Roberts, et al.).

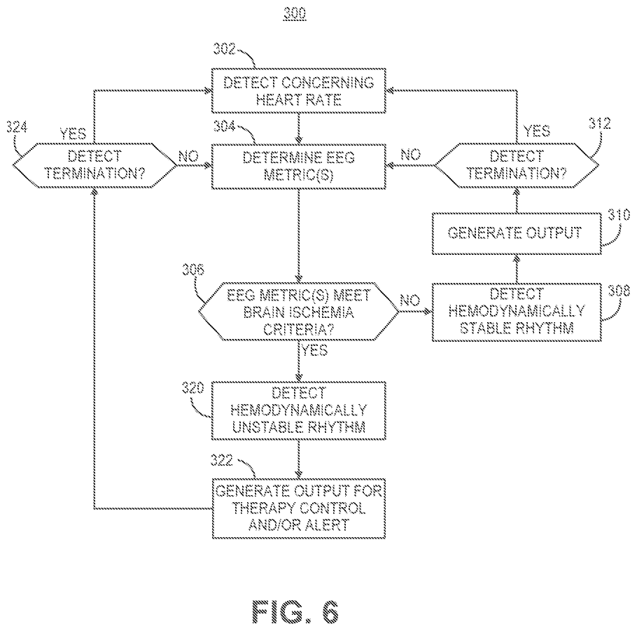

FIG. 6 is a flow chart 300 of a method that may be performed by a medical device system for discriminating between HU and HS cardiac rhythms according to some examples. The process of FIG. 6 may be performed by processing circuitry included in one or more IMDs, e.g., an EEG sensor 40 and one or more of ICD 14, ICD 114, cardiac monitor 44 and/or pacemaker 150. For the sake of illustration, the process of FIG. 6 is described in conjunction with an IMD system including a co-implanted EEG sensor 40 and ICD 14 as shown in FIG. 1. However, it is to be understood that a different combination of medical devices, which may include one or more wearable or other external devices, may be configured to cooperatively detect a concerning heart rate and identify the cardiac rhythm as either HU or HS. Furthermore, in some examples, a single device may sense both electrical cardiac activity and electrical brain activity for detecting HU cardiac rhythms and generating an alert or EMS call and/or delivering a therapy to promote or restore hemodynamic stability.

At block 302, a concerning heart rate can be detected from an ECG signal sensed by ICD 14. The concerning heart rate may be a fast heart rate detected based on RRIs determined from ventricular sensed event signals received by control circuit 80 from sensing circuit 86, for example. A fast heart rate may be detected based on a threshold number of RRIs that are shorter than a fast rate threshold out of a most recent number RRIs. A fast heart rate may be detected, for example, when the NID is reached for detecting VT. In other examples, the fast heart rate may be detected when fewer VT intervals are counted by control circuit 80 than the NID required to detect VT. In other examples, the fast heart rate may be detected when a threshold number of fast VT or VF intervals is reached, where the fast VT or VF detection intervals are shorter than VT detection intervals. Accordingly, detection of a fast heart rate may be detection of VT or VF associated with the fast heart rate according to tachyarrhythmia detection algorithms in some examples. However, in other examples, detection of a fast heart rate as a concerning heart rate at block 302 may not correspond to VT or VF detection criteria being met and may be based on different interval/rate based criteria than VT or VF detection criteria.

With no limitation intended, when a threshold number of intervals that are shorter than 400 ms, 380 ms, 360 ms, 340 ms, 320 ms, 300 ms, 280 ms, or 260 ms, control circuit 80 may detect a fast heart rate at block 302. The threshold number may be between 3 RRIs and 100 RRIs, as examples. In some examples, the threshold number of intervals is between 12 RRIs and 80 RRIs. In some examples, a relatively high threshold number of RRIs may be applied to detect the fast heart rate and initiate EEG signal analysis because the EEG signal changes due to brain ischemia caused by an HU cardiac rhythm can lag the onset of the fast heart rate. In other examples, the EEG signal analysis may begin after a relatively small threshold number of RRIs less than a fast rate threshold interval. Initiating EEG signal monitoring early in the onset of a fast heart rate may enable the time of onset and the time interval over which changes in the electrical brain activity occur to be tracked. The time of onset and the time over which changes in the electrical brain activity occur may enable the medical device system to discriminate between brain ischemia due to a HU cardiac rhythm and other conditions that may cause similar EEG signal changes, such as sleep, pharmaceutical effects, stroke or the like. Accordingly, in various examples, the threshold number of RRIs that are required to be less than a fast interval threshold may be less than, equal to, or greater than an NID used by control circuit 80 for detecting VT or VF. In other examples, a mean, median or other representative RRI may be determined from a specified number of RRIs, e.g., 8 to 100 RRIs, and compared to a fast interval threshold for detecting a fast heart rate.

In response fast heart rate criteria being met, analysis of the EEG signal may be performed to discriminate the cardiac rhythm as being HS or HU. In other examples described below, before analyzing the EEG signal for discriminating between HS and HU cardiac rhythms when a fast heart rate is detected, the ICD control circuit 80 may detect a tachyarrhythmia based on VT or VF detection criteria being met based on morphology analysis of one or more sensed ECG (or EGM) signals in addition to or alternatively to interval-based rate criteria.

In some instances, the concerning heart rate detected at block 302 may be a slow or irregular heart rate detected based on RRIs determined from ventricular sensed event signals received by ICD control circuit 80 from sensing circuit 86. A slow heart rate may be detected based on a threshold number of RRIs that are longer than a slow rate threshold interval out of a most recent number RRIs. A slow heart rate may be detected when a mean, median or other representative RRI is longer than a slow rate threshold interval. In other examples, an irregular heart rate may be detected as a concerning heart rate, e.g., based on RRI variability. A slow heart rate may be equal to or faster than a programmed lower pacing rate. For example, a programmed lower pacing rate may be 30, 40, or 50 beats per minute (bpm). A concerning heart rate may be detected in a given patient when the RRIs correspond to a heart rate that is less than 60 bpm, 55 bpm, 50 bpm, 45 bpm, 40 bpm, 35 bpm or 30 bpm as examples. Some patients may experience various forms of syncope that could occur at relatively slow heart rates, which may be faster than a programmed lower pacing rate (and in some cases the implanted device(s) of the medical device system may not be configured to deliver bradycardia pacing). The medical device system, therefore, may be configured to detect a concerning heart rate according to fast heart criteria and/or slow heart criteria in accordance with a particular patient's individual needs.

In some examples, upon detecting a concerning heart rate at block 302, control circuit 80 may cause communication circuit 88 to transmit a signal to EEG sensor 40 requesting brain ischemia data. In other examples, EEG sensor 40 may be configured to detect the concerning heart rate using any of the criteria described above so that a transmitted request for brain ischemia data from ICD 14 is not required to trigger EEG sensor 40 to analyze the electrical brain activity. As described above, EEG sensor 40 may be configured to extract electrical cardiac activity from the electrical signal received via electrodes 42. For example, EEG sensor 40 may filter the electrical signal, use blind source separation or other techniques to obtain an ECG signal (or more generally electrical cardiac activity) from which the concerning heart rate can be detected at block 302.

When EEG sensor 40 detects a concerning heart rate, EEG sensor 40 may analyze the sensed EEG signal to determine EEG metrics at block 304, without necessarily requiring receipt of a transmitted request from ICD 14. Accordingly, in some examples, both EEG sensor 40 and ICD 14 may be configured to detect the concerning heart rate at block 302 from electrical signals sensed by the respective devices. In still other examples, only EEG sensor 40 may detect the concerning heart rate at block 302 based on RRI data determined from an ECG signal filtered from the electrical signal sensed by EEG sensor 40. ICD 14 may be performing VT/VF or other arrhythmia detection algorithms that may or may not include detecting the concerning heart rate according to the same interval- or rate-based criteria and thresholds used by EEG sensor 40. In this case, EEG sensor 40 may transmit data to ICD 14 for use in determination of a HU cardiac rhythm if needed by ICD 14, e.g., when VT detection criteria are met.

At block 304, processing circuit 200 of EEG sensor 40 may determine one or more EEG metrics from the sensed EEG signal. The EEG metrics may be determined in response to the EEG sensor 40 detecting the concerning heart rate or in response to a communication signal received from ICD 14. At block 306, EEG sensor processing circuit 200 may determine if the EEG metric(s) meet brain ischemia criteria. When brain ischemia criteria are not met, the medical device system may detect an HS cardiac rhythm at block 308. For example, EEG sensor 40 may transmit a signal at block 308 indicating no brain ischemia detected or “normal” EEG metrics and/or an HS cardiac rhythm indicator. Processing circuitry of the medical device system may generate an output at block 310 in response to detecting the HS cardiac rhythm. The output may be used for controlling a response to the HS cardiac rhythm detection. For example, in response to receiving a transmitted signal from EEG sensor 40 indicating no brain ischemia, ICD control circuit 80 may generate an output, which may be stored in memory 82, that can include a therapy selection and/or therapy control signal(s) for use in selecting and controlling a therapy response at block 310. The therapy response may include delivering, withholding or delaying a therapy, depending on the detected heart rate or detected arrhythmia and the programmed therapies available. The output generated at block 310 may be used to control therapy delivery circuit 84 for delivering, withhold or delaying a therapy, depending on the detected heart rate and cardiac rhythm. In other examples, the output generated at block 310 may be used in transmitting a therapy control signal to another medical device, e.g., pacemaker 150 or a neurostimulation device.

When the concerning heart rate is detected as a fast heart rate, ICD control circuit 80 may generate a therapy control signal output at block 310 to withhold all ATP and CV/DF therapies because the fast heart rate is determined to be HS (brain ischemia criteria not met at block 306). In other examples, control circuit 80 may select the therapy response at block 310 by controlling therapy delivery circuit 84 to deliver ATP when VT is detected by control circuit 80 but the fast heart rate associated with the VT is determined to be an HS cardiac rhythm. Control circuit 80 may withhold or delay any CV/DF shock therapy that may be programmed in a menu of therapies when VT is detected if the VT is HS. In still other examples, when pacemaker 150 is present, a communication signal may be transmitted from EEG sensor 40 or from ICD 14 to trigger pacemaker 150 to deliver ATP in response to detecting a fast heart rate that is HS. ICD control circuit 80 may withhold or delay a CV/DF shock in response to detection of VT that is determined to be HS.

When the concerning heart rate is a slow heart rate that is determined to be HS, therapy delivery circuit 84 of ICD 14 (or pacemaker 150 if present) may be controlled (based on output generated at block 310) to withhold cardiac pacing. However, if the slow heart rate falls below a programmed lower pacing rate, the therapy delivery circuit 84 (or pacemaker 150) may deliver cardiac pacing at the lower rate to treat bradycardia and prevent asystole or long ventricular pauses.

Output generated at block 310 may include storage of an EEG signal episode and/or ECG signal episode and/or other sensor signal episodes that may be available. The output may include EEG metrics and or a brain ischemia stage determined from the EEG metrics. Data stored in memory of the medical device system in conjunction with an HS cardiac rhythm determination may be transmitted to external device 50 for use by a clinician in monitoring and managing the patient.

ICD control circuit 80 and/or EEG sensor processing circuit 200 may be configured to detect termination of the concerning heart rate at block 312. For example, termination of a fast heart rate may be detected based on a threshold number of slow RRIs that are longer than the fast rate interval or longer than the fast rate interval plus an offset. The threshold number of slow RRIs required to detect termination may be equal to, less than or greater than the threshold number of fast RRIs required to detect the fast heart rate at block 302. ICD 14 may be configured to detect termination of VT at block 312 according to a number of termination detection algorithms and criteria. When the concerning heart rate is detected as a slow heart rate, termination may be detected at block 312 when the heart rate is greater than a slow heart rate threshold, e.g., based on a threshold number of RRIs or a mean or median RRI being shorter than a slow rate RRI threshold. When termination of the concerning heart rate is detected, the process of flow chart 300 may return to block 302 to wait until concerning heart rate criteria are met again. If termination is not detected, EEG sensor processing circuitry 200 may re-determine EEG metrics at block 304 for comparison to brain ischemia criteria at block 306.

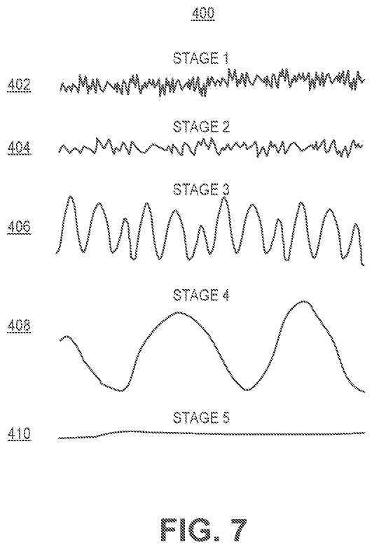

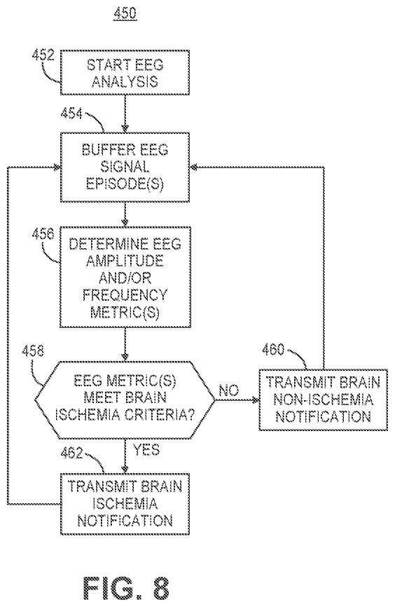

When the EEG metric(s) meet brain ischemia criteria at block 306 that indicate an HU cardiac rhythm, the medical device system detects the HU cardiac rhythm at block 320. Examples of EEG metrics and criteria for detecting brain ischemia, and optionally discriminating between different stages of brain ischemia, are described below, e.g., in conjunction with FIGS. 7 and 8. The HU cardiac rhythm may be detected at block 320 by ICD control circuit 80 in response to a communication signal received from EEG sensor 40, transmitted when the EEG metrics meet brain ischemia criteria as determined by EEG sensor processing circuitry 200. In these examples, EEG sensor processing circuitry 200 determines when EEG metric(s) meet brain ischemia criteria and controls EEG sensor communication circuit 208 to transmit a signal indicating that brain ischemia is (or is not) detected. It is to be understood that in other examples, EEG sensor 40 may be configured to transmit determined EEG metrics to ICD 14. ICD control circuit 80 may analyze the EEG metrics for determining when brain ischemia is detected, which may include determining a stage of brain ischemia as further described below. In still other examples, EEG sensor 40 may transmit an EEG signal episode to ICD 14 for determining EEG metrics, detecting a stage of brain ischemia and determining whether a detected concerning heart rate is HS or HU.

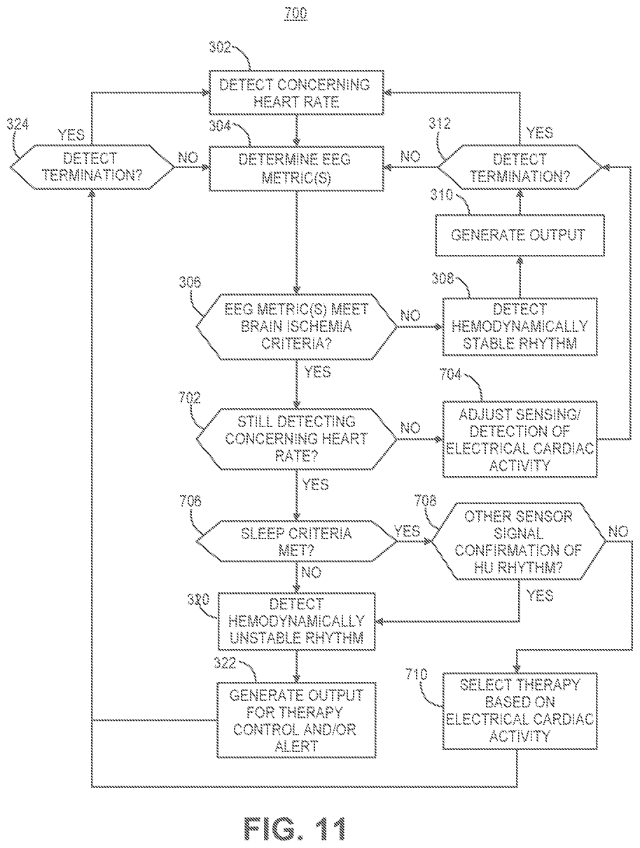

When a concerning heart rate is detected and either slows (from a fast rate) or increases (from a slow rate) to a rate that is not a concerning heart rate, EEG analysis may be aborted. For example, ICD 14 may transmit a signal to EEG sensor 40 to indicate that a concerning heart rate is no longer being detected at any time during the process of flow chart 300. Spontaneous termination of a non-sustained VT could occur, for example, in which case EEG signal analysis may no longer be required. Therefore, while blocks 312 and 324 for detecting termination are shown to occur after generating an output at block 312 or 322 for providing a response to an HS cardiac rhythm detection or an HU cardiac rhythm detection, respectively, it is to be understood that termination of a concerning heart rate may be determined by ICD control circuit 80 (and/or EEG sensor 40) at any time during the process of flow chart 300. When termination of the concerning heart rate is detected, EEG analysis for determining brain ischemia may be aborted. In other examples, however, EEG analysis for determining brain ischemia may continue to verify hemodynamic recovery after termination of a concerning heart rate, e.g., as described below in conjunction with FIG. 11. In some cases, termination of a concerning heart rate may be falsely detected, e.g., due to undersensing or oversensing of cardiac electrical event signals.

When an HU cardiac rhythm is detected at block 320 in response to a concerning heart rate and brain ischemia detection, the processing circuitry of the medical device system may generate an output for providing a response to detecting the HU cardiac rhythm at block 322. The output may be stored in memory of the medical device system, e.g., received and stored by memory 82 from control circuit 80, and used by the medical device system to perform one or more responses at block 322. The output may include a command or signal for transmitting an alert or notification of the HU cardiac rhythm. The output may be used to transmit a patient, clinician, caregiver or first responder alert by the medical device system, e.g., by EEG sensor communication circuit 208 or by ICD communication circuit 88. External device 50 or another device may receive the alert and be configured to make a phone call to EMS. Transmission of an alert indicating detection of a HU cardiac rhythm may be the primary response performed by a medical device system that includes sensing and monitoring devices without therapy delivery capabilities (or with only cardiac pacing therapy capability but without CV/DF shock therapy capability), e.g., when EEG sensor 40 and cardiac monitor 44 are co-implanted in a patient with or without pacemaker 150.

Additionally or alternatively, the output generated by the medical device system processing circuitry at block 322 may include a therapy selection signal or therapy control signal for providing a response at block 322 that includes delivering a therapy by a medical device of the medical device system. The generated output may include therapy control signal that is a CV/DF shock therapy signal. ICD 14 may deliver a CV/DF shock upon determination of an HU cardiac rhythm, particularly when VT or VF detection criteria are met. In some examples, ATP may be delivered before a CV/DF shock is delivered in a first attempt at terminating the HU cardiac rhythm. ATP may be delivered if the brain ischemia has not reached a moderate or severe stage, e.g., associated with cell injury or death, or ATP may be delivered during high voltage capacitor charging in an attempt to treat the HU cardiac rhythm as quickly as possible, before capacitor charging for CV/DF shock delivery is complete. In general, one or more CV/DF shocks may be delivered as early as possible after detecting an HU cardiac rhythm to avoid or minimize the duration of loss of consciousness and/or avoid or minimize ischemic brain injury and cell death.

When the concerning heart rate is a slow heart rate determined to be HU, the response at block 322 may include generating therapy pacing control signal(s) to control delivery of cardiac pacing at a rate faster than the sensed heart rate, according to the therapy control signal(s) to promote or restore hemodynamic stability. The pacing rate may be faster than a programmed lower rate in order to pace the heart at a rate faster than the intrinsic heart rate detected as an HU cardiac rhythm. The output generated by control circuit 80 to provide a response to detecting an HU cardiac rhythm at block 322 may include pacing control signal(s) that can be passed to therapy delivery circuit 84 for controlling cardiac pacing. The output generated by control circuit 80 to provide a response to detecting an HU cardiac rhythm at block 322 may include a pacing control signal that is transmitted to another device, e.g., to pacemaker 150, to deliver cardiac pacing. In examples of medical device systems that include neurostimulation therapy capabilities, a therapy control signal may be output at block 322 to cause stimulation of the vagus nerve, spinal cord or other nervous system tissue delivered to alter the autonomic tone, e.g., increase the sympathetic nervous system activity.

The output generated at block 322 may include an adjustment to the programmed lower pacing rate when the HU cardiac rhythm is a slow or variable heart rate that is faster than the currently programmed pacing lower rate. The programmed lower pacing rate may be incremented toward or adjusted to the pacing rate required to overdrive pace the intrinsic heart rate detected as a HU cardiac rhythm. The programmed lower pacing rate may be increased to be greater than the detected concerning heart rate by 5 to 20 bpm or about 10 bpm faster than the intrinsic, concerning heart rate. In this way, the current episode of the HU cardiac rhythm may be treated by delivering cardiac pacing according to an adjusted lower pacing rate. Future episodes of an HU cardiac rhythm that is a slow or variable heart rate may be avoided because pacing at the adjusted lower pacing rate may avert an HU cardiac rhythm.

The increased lower pacing rate may be a temporary lower pacing rate that may be in effect for a fixed period of time, which may be programmable. The increased temporary lower pacing rate may remain in effect for 0.2, 0.5, 1, 4, 8, 12, or 24, 48, or 72 hours, as examples with no limitation intended. The lower pacing rate may return to the programmed permanent lower pacing rate when the time period expires. In some examples, the EEG signal continues to be monitored during and/or after pacing at the temporary lower pacing rate to verify that the brain ischemia condition is reversed and, if not, an EMS call or other urgent notification or alert signal can be transmitted by ICD 14 or EEG sensor 40. If the brain ischemia condition is still being detected during the time period, the temporary lower pacing rate could be increased, e.g., by 5 to 10 bpm, up to a specified maximum lower pacing rate in an attempt to alleviate the brain ischemia condition. If the brain ischemia criteria are met after the time period expires, pacing at the temporary lower pacing rate may be resumed for another time period, and the time period may optionally be increased.

In other examples, the permanent lower pacing rate may be increased in response to detecting an HU cardiac rhythm that is a slow or variable heart rate. The output generated at block 322 may include a notification transmitted to external device 50 to notify a clinician that the permanent (or temporary) lower pacing rate has been increased and to what rate. A clinician may reprogram the permanent lower pacing rate as needed. For instance, the clinician may choose to reprogram the permanent lower pacing rate to the automatically adjusted temporary lower pacing rate.

ICD control circuit 80 may generate the output at block 320 by storing the adjusted temporary or permanent lower pacing rate in memory 82. When another co-implanted device, e.g., pacemaker 150, is present for providing bradycardia pacing, control circuit 80 may control communication circuit 88 to transmit a command to adjust the lower pacing rate, either temporarily or permanently. Pacemaker 150 may receive the command from ICD 14, directly or via external device 50, and adjust the permanent or temporary lower pacing rate accordingly.

At block 324, the EEG sensor 40 and/or the ICD 14 may be configured to determine if the HU cardiac rhythm has been terminated by the delivered therapy. In some examples, if a fast cardiac rhythm has not been terminated following ATP or a CV/DF shock, control circuit 80 may control ICD therapy delivery circuit 84 to deliver another therapy, without necessarily re-evaluating the EEG signal for detecting brain ischemia. In other examples, the EEG signal may continue to be analyzed by EEG sensor processing circuit 200 while ICD 14 is delivering one or more therapies to determine if a detected brain ischemia condition is improved or no longer being detected. If an improvement in brain ischemia is detected, e.g., such that brain ischemia criteria for detecting an HU cardiac rhythm are no longer met at block 306, EEG sensor communication circuit 208 may transmit a signal to ICD 14 to indicate that brain ischemia is no longer detected. The medical device system may detect an HS cardiac rhythm at block 308 and may withhold or delay any scheduled therapies at block 310. For example, if a first CV/DF shock has been delivered and the fast rate has not been terminated but the EEG metric(s) no longer meet brain ischemia criteria, a next scheduled CV/DF shock may be withheld at block 310. ATP may be delivered instead if VT detection criteria are still being met. In this way, EEG sensor 40 and an ICD (and/or pacemaker), e.g., ICD 14 or ICD 114 (and/or pacemaker 150), may control ATP and CV/DF therapies based on whether a detected concerning heart rate is identified as an HS or HU cardiac rhythm based on EEG metrics.

When the concerning heart rate is a slow heart rate, control circuit 80 may determine if the intrinsic heart rate is increased at block 324 (e.g., by withholding cardiac pacing by therapy delivery circuit 84 and determining intrinsic RRIs are shorter than a slow heart rate interval threshold). If the intrinsic heart rate is increased, the process may return to block 302. However, when the concerning heart rate is a slow heart rate, the medical device system may be configured to determine if EEG metrics no longer meet brain ischemia criteria at block 324 for detecting termination of the HU cardiac rhythm after starting cardiac pacing.

Regardless of the intrinsic heart rate after cardiac pacing is started in response to a slow heart rate that is HU, the medical device system may verify that brain ischemia is no longer detected, particularly if cardiac pacing is stopped. The cardiac pacing may continue and the EEG metrics may be re-determined at block 324 for detecting termination of the HU cardiac rhythm. In other examples, the cardiac pacing may be terminated and the EEG metrics may be re-determined at block 324 for detecting termination of the HU cardiac rhythm. If brain ischemia is still being detected such that the HU cardiac rhythm is not terminated (“no” branch of block 324), cardiac pacing may be restarted if EEG metrics meet brain ischemia criteria again (as determined at blocks 304 and 306). Cardiac pacing started in response to detecting a slow heart rate that is HU at block 322 may be delivered for a fixed interval of time, e.g., 30 seconds to one hour of cardiac pacing, or for a fixed number of cardiac pacing pulses, e.g., 10 to 100 pacing pulses. After the specified cardiac pacing time interval is expired (or specified number of pacing pulses delivered), the EEG metrics may be redetermined at block 324 to verify that the HU cardiac rhythm is terminated (whether or not a slow heart rate is still being detected).

FIG. 7 is a diagram 400 of various examples of EEG signals 402, 404, 406, 408 and 410 that may be sensed by EEG sensor 40 during varying stages (1 through 5) of brain ischemia. Cerebral perfusion can be measured in blood volume, e.g., milliliters (mL), per unit mass of brain tissue per unit time, and may typically average about 50 mL/ 100 grams/minute. It is recognized, however, that during normal physiological conditions, different portions of the brain (e.g., white matter vs. gray matter) may have higher or lower perfusion rates than this general average when blood flow to the brain is unimpaired. Cerebral blood flow (CBF) can be measured as blood volume per unit time. CBF can typically be 700 to 800 or about 750 mL/minute and can be about 10 to 20% or about 15% of the cardiac output.

The EEG power spectrum representative of electrical brain activity can include multiple frequency bands referred to as beta, alpha, delta and theta frequency bands. Beta waves can be defined as neural oscillations of the EEG power spectrum that are 12 Hz or higher, e.g., in the 12 Hz to 38 Hz range, in the 14 Hz to 30 Hz range or in the 15 Hz to 40 Hz range as examples. Alpha waves are neural oscillations in a range that can be defined by a lower limit of about 6 to 8 Hz to an upper limit of about 12 Hz to about 15 Hz. Theta waves are neural oscillations that can be in a range defined by a lower limit of about 3 Hz to 5 Hz to an upper limit of about 6 Hz to about 8 Hz. Delta waves are neural oscillations that can be in a range of frequencies less than 5 Hz, less than 4 Hz, less than 3 Hz or less than 2.5 Hz. The delta band may be defined having a lower limit of 0.5 to 1 Hz. As brain ischemia progresses with decreasing cerebral perfusion, the EEG power spectrum may shift from a relatively higher predominant frequency band to a relatively lower predominant frequency band to total or near total electrical brain activity suppression as shown by EEG signal 410 in stage 5 brain ischemia.

Beta waves are present in the EEG during normal waking consciousness, during normal brain perfusion, and during initial, relatively small decreases (e.g., less than 30% decrease) in brain perfusion. EEG signal 402 represents a normal EEG signal during normal CBF or a stage 1 ischemia during waking consciousness, when CBF may be reduced but without significant changes occurring in electrical brain activity compared to normal electrical brain activity. The dominant frequency of EEG signal 402 may be in the beta band, e.g., when cerebral perfusion is still within 80%, 75% or even 70% of a normal perfusion rate. EEG signal 402, therefore, illustrates the EEG signal that may be sensed by EEG sensor 40 during normal CBF or during “stage 1” of 5 possible stages of brain ischemia. CBF or cerebral perfusion may be reduced less than 30%, reduced less than 25% or reduced less than 20% from a normal average cerebral perfusion rate for the given patient or a normal average cerebral perfusion rate representative of a population of patients. Stage 1 may be detected by processing circuit 200 of EEG sensor 44, for example, based on EEG metrics determined from EEG signal 402 that are within a normal range of the analogous EEG metrics determined from an EEG signal sensed during normal, non-impaired CBF conditions in an awake patient.

A threshold based on a normal value of an EEG metric representative of a population of patients may be programmed in EEG sensor memory 202 for comparison to an EEG metric that is determined by processing circuit 200 from sensed electrical brain activity. In other examples, a threshold applied to an EEG metric for detecting a stage of brain ischemia may be tailored to the patient. For instance, as further described below, EEG sensor processing circuit 200 may establish baseline or normal EEG metric(s) for a given patient by determining EEG metrics during a normal sinus rhythm, e.g., heart rate in a normal resting range such as less than 100 bpm and greater than at least 40 bpm or in the range of about 60 to 90 bpm. The normal resting range may be confirmed by EEG sensor 40 by determining a heart rate from ECG data extracted from a signal sensed from EEG sensor electrodes 42 and/or based on a communication signal transmitted by another device, e.g., external device 50, cardiac monitor 44, ICD 14, ICD 114 or pacemaker 150. The normal resting range for a given patient may be programmable by a user using external device 50 in some examples or may be “learned” by the medical device system based on tracking a history of heart rates for the given patient.

The EEG signals 404, 406, 408 and 410 represent increasing (worsening) stages of brain ischemia as cerebral perfusion decreases, e.g., due to an HU cardiac rhythm. The higher frequency content, e.g., in the beta band, that is present in EEG signal 402 is decreased in EEG signal 404. The dominant frequency in EEG signal 404 may be in the alpha band, e.g., in the range of about 7 to 15 Hz or about 8 to 14 Hz, as examples. The stage 2 EEG signal 402 may correspond to cerebral perfusion that is reduced by 30% to 50% from normal cerebral perfusion. For instance, the stage 2 EEG signal may be sensed by EEG sensor 40 during cerebral perfusion in the range of about 25 to 35 mL/100 g/minute.

EEG signal 406 represents the EEG signal that may be sensed by EEG sensor 40 during a further decrease in cerebral perfusion, e.g., a decrease of 50% to 70% in CBF, or in the range of about 18 to 25mL/100 g/minute. A further loss in higher frequency content can occur with increased brain ischemia represented by stage 3 in FIG. 7. The dominant frequency during stage 3 may be in the theta band, e.g., between 4 and 7 Hz. In addition to a decrease in higher frequency band content in the EEG signal during ischemia, it can be observed from FIG. 7 that the amplitude of the electrical brain activity oscillations can increase. As such, EEG sensor processing circuit 200 may be configured to determine a frequency metric and/or an amplitude metric from a sensed EEG signal for determining a stage of brain ischemia.

With a further decrease in cerebral perfusion, the EEG signal 408 shown as representative of stage 4 brain ischemia may be sensed by EEG sensor 40. During stage 4 brain ischemia, the dominant frequency of EEG signal 408 may be in the delta band, e.g., between 0.5 Hz and 4 Hz. Stage 4 brain ischemia with dominant delta band frequencies can be associated with the onset of ischemic brain injury. The amplitude of the oscillations of EEG signal 408 are further increased compared to lower stages 1-3. In stage 5 brain ischemia, suppression of neural oscillations occurs. During stage 5 brain ischemia, cell death and brain infarct can occur. In some examples, a threshold for detecting an HU cardiac rhythm versus an HS cardiac rhythm may correspond to detecting stage 2 or stage 3 brain ischemia. Detection of stage 2 or higher or detection of stage 3 or higher may be evidence of a HU cardiac rhythm, e.g., when a concerning heart rate is detected. Detection of stage 1 EEG metrics may be indicative of an HS cardiac rhythm.

In a general example, when one or more amplitude metrics and/or one or more frequency metrics determined from the electrical brain activity indicate stage 1 brain ischemia, a HS cardiac rhythm may be detected by the medical device system. If the cardiac rhythm is a fast heart rate, which may be associated with VT or VF, a CV/DF shock may be withheld or delayed when the medical device system is capable of delivering high voltage shocks. If one or more amplitude metrics and/or one or more frequency metrics determined from the electrical brain activity indicate moderate brain ischemia, e.g., stage 2, 3 or 4, a concerning heart rate may be detected as a HU cardiac rhythm by the medical device system. Detection of the moderate brain ischemia may be based on a relatively increased amplitude and/or relatively decreased frequency of the neural oscillations in the sensed electrical brain activity. When the cardiac rhythm is a fast heart rate that may be associated with VT or VF, a CV/DF shock may be delivered to terminate the HU cardiac rhythm. When the sensed electrical brain activity is determined to be severe ischemia, e.g., stage 5, a CV/DF shock may or may not be delivered in various examples.

For instance, a limited number of CV/DF shocks may be delivered, e.g., one to eight, when a HU cardiac rhythm is detected based on moderate brain ischemia criteria and a fast heart rate. However, when stage 5 brain ischemia is detected, the patient may experience extensive irreversible brain damage or be clinically dead. Delivery of CV/DF shocks may be futile. As such, when stage 5 brain ischemia (e.g., suppression or disappearance of neural oscillations) is detected based on analysis of the sensed electrical brain activity by the medical device system, CV/DF shocks are not delivered by the medical device system in some examples. Particularly if one or more CV/DF shocks have already been delivered in response to detecting the HU cardiac rhythm prior to detecting stage 5 brain ischemia, e.g., while stage 2, 3, or 4 is being detected, any additional CV/DF shocks scheduled or programmed to occur in a menu of therapies may be aborted. If stage 5 brain ischemia is reached, cardiac electrical stimulation therapy for terminating the HU cardiac rhythm may be aborted in some examples.

FIG. 8 is a flow chart 450 of a method for detecting brain ischemia for use in classifying a cardiac rhythm as being HU or HS according to some examples. The process of flow chart 450 is described as being performed by EEG sensor 40 for the sake of illustration. However, it is to be understood that in some examples, the EEG signal may be extracted from a cardiac electrical signal sensed by cardiac monitor 44 or another IMD or transmitted from EEG sensor 40 to another co-implanted IMD or an external device, e.g., external device 50, for analysis for detection of brain ischemia.

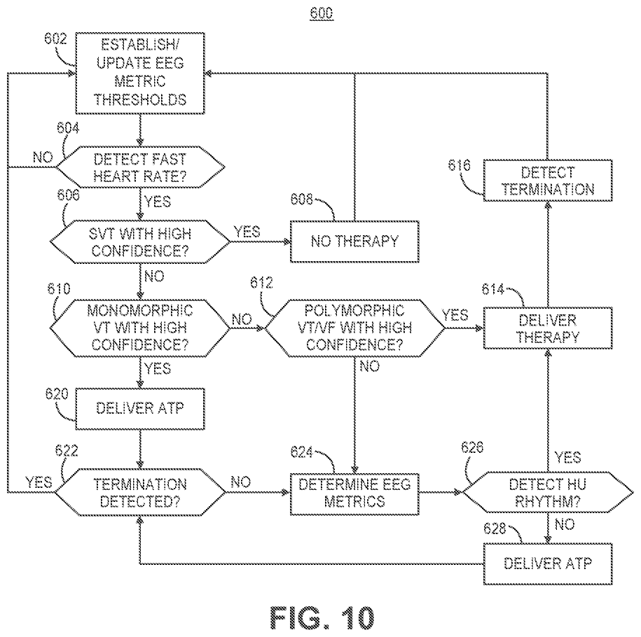

At block 452, EEG sensor 40 may initiate EEG analysis for determining EEG metrics for use in discriminating between HU and HS cardiac rhythms. In some examples, EEG sensor 40 starts the process of flow chart 450 in response to a communication signal received from a co-implanted IMD that has detected a concerning heart rate. As described above, the concerning heart rate may be a heart rate may be a fast heart rate that meets fast heart rate criteria, a slow heart rate that meets slow heart rate criteria, or a variable heart rate that meets variable heart rate criteria. In some examples, the communication signal may be transmitted by a co-implanted IMD when a monomorphic VT is detected or when a tachyarrhythmia is detected without a high degree of confidence (as further described below in conjunction with FIG. 10). In other examples, EEG sensor 40 may filter or extract cardiac electrical activity from the electrical signal sensed using electrodes 42 and determine a heart rate from the cardiac electrical activity. If a fast heart rate is detected, e.g., greater than 120 bpm, greater than 140 bpm, greater than 160 bpm, greater than 180 bpm, or greater than 200 bpm as examples, EEG sensor 40 may start EEG analysis for detecting brain ischemia, without requiring a command or request received from another medical device. If a slow heart rate is detected, e.g., less than 60 bpm, less than 55 bpm, less than 50 bpm, or less than 40 bpm, the EEG sensor 40 may start EEG analysis. In still other examples, EEG sensor 40 may be configured to monitor the EEG signal on a continuous basis or intermittently, e.g., at regular time intervals such as every 30 seconds, once per minute, or other scheduled basis. In this case, processing circuit 200 may store an EEG signal episode in memory 202 in a rolling manner to enable processing circuit 200 to determine EEG metrics at regular time intervals for detecting a change in the EEG metrics that is indicative of brain ischemia.

At block 454, an EEG signal episode may be stored in memory 202 for determining EEG metrics by processing circuit 200. In some examples, the EEG signal episode buffered in memory 202 may begin upon receiving a communication signal requesting an EEG analysis from another device or upon detecting a concerning heart rate by processing circuit 200. In other examples, the EEG signal episode may begin at a time delay after a concerning heart rate is detected, a tachyarrhythmia detection is made or a communication signal is received requesting an EEG analysis. EEG signal changes associated with brain ischemia may be delayed in time from the onset of an HU cardiac rhythm. As such, the EEG signal episode may be buffered in memory 202 starting 5 seconds, 10 seconds, 15 seconds, 20 seconds or 30 seconds after a triggering event (e.g., fast heart rate detection or VT detection) that starts the EEG signal analysis. Additionally or alternatively, as described above, an EEG signal episode may be stored in memory 202 on a rolling basis so that electrical brain activity can be monitored independently of heart rate or a received request. In this way, if brain ischemia is detected, a notification may be transmitted by EEG sensor 40 for use by a co-implanted device in determining if a therapy response is warranted based on the notification and analysis of electrical cardiac activity.

The EEG signal episode may extend for a specified time duration, e.g., between 0.25 and sixty seconds or between about 1 and 5 seconds in various examples. In some examples, multiple EEG signal episodes may be buffered over a desired monitoring time period, each for a specified time duration, instead of buffering one relatively longer EEG signal episode. The multiple EEG signal episodes may each be 0.25 to 6 seconds in duration, for example, and be buffered 0 to 30 seconds apart for a total monitoring period of several seconds, several minutes, or up to one hour, for instance. To illustrate, upon starting the EEG analysis process of flow chart 450, processing circuit 200 may buffer EEG signal episodes in memory 202 that are each 0.5 to 3 seconds in duration every 5 seconds to 60 seconds until a concerning heart rate is no longer detected by the medical device system or until termination criteria are met after detecting VT or VF or other cardiac arrhythmia.

At block 456, processing circuit 200 determines one or more EEG metrics from each of the one or more EEG signal episodes buffered in memory 202. In some examples, processing circuit 200 determines one or more EEG amplitude metrics. Example amplitude metrics may include an absolute maximum peak amplitude of the entire EEG signal episode, an average or median of all signal peaks during the EEG signal episode, an average amplitude of all sample points spanning the rectified EEG signal episode, or an average amplitude of all sample points having a rectified amplitude that is greater than a threshold amplitude during the EEG signal episode. One or more EEG amplitude metrics may be determined to detect a decrease in the amplitude of neural oscillations in the EEG signal that can occur as CBF decreases due to decreased cardiac output during an HU cardiac rhythm. See for example the relative increase in amplitude in the neural oscillations in stage 3 compared to stage 2 or stage 4 compared to stage 3 in FIG. 7.

Additionally or alternatively, processing circuit 200 may determine one or more EEG frequency metrics at block 456. A frequency metric may be determined in the frequency domain or in the time domain. For example, a Fourier transform or other time to frequency domain transform method may be performed by processing circuit 200 to determine the frequency content of the EEG signal episode. In some examples, the dominant frequency, center frequency, mean frequency, median frequency or other representative frequency may be determined as a frequency metric of the EEG signal episode. Additionally or alternatively, the power of each of the beta band, alpha band, theta band and delta band of EEG signal frequencies may be determined from the EEG signal episode after performing a Fourier transform. A quantitative or qualitative relationship between the frequency bands may be determined for classifying the EEG signal episode as being in one of stages 1 to 5 as generally shown in FIG. 7.

For instance, a shift in a dominant frequency band from a previously determined dominant frequency band of a preceding EEG signal episode, or a shift from a dominant frequency in the beta band to the alpha band or from the alpha band to the theta band, as examples, may be detected as a change in the stage of brain ischemia. For instance, at block 458, processing circuit 200 may determine if the EEG metrics meet brain ischemia criteria by comparing the dominant frequency determined from the EEG signal episode to each of multiple frequency ranges defining the frequency bands associated with each stage of brain ischemia as described in conjunction with FIG. 7. When the dominant frequency is lower than the beta band, for instance, brain ischemia criteria may be met.

In other examples, time domain frequency metrics may be determined, which may reduce the required processing burden and time and current drain of EEG sensor power source 210 compared to calculations made in the frequency domain for determining an EEG frequency metric. For instance, a frequency metric may be determined as a number of oscillations during the EEG signal episode. The number of oscillations may be determined by counting the number of zero (or other threshold) crossings, counting the number of peaks, or counting signal inflections, as examples. The number of oscillations determined during an EEG signal episode of a specified time duration can be compared to different thresholds, each corresponding to a lower limit of a frequency band of each of the stages of brain ischemia described above. Processing circuit 200 may determine the brain ischemia stage at block 458 based on the number of oscillations in the EEG signal episode of a specified time duration. For example, processing circuit 200 may determine brain ischemia criteria are met when the number of oscillations counted in an EEG signal episode of a specified time duration is less than a threshold number.

Another example of a frequency metric that may be determined in the time domain by processing circuit 200 involves determining a derivative or difference signal from the EEG signal episode. An EEG frequency metric may be determined as a maximum peak of the derivative signal, an average, median or other representative value of peak amplitudes of the derivative signal, or an average or other quantitative relationship of the amplitude differences between the derivative signal and the original EEG signal.

At block 458, processing circuit 200 may compare the EEG metric(s) determined at block 456 to criteria for detecting brain ischemia. In some examples, the stage of brain ischemia is determined based on a frequency and/or amplitude metric and when the stage is greater than a threshold stage for detecting brain ischemia, the EEG sensor communication circuit 208 may transmit a brain ischemia notification at block 462. When the stage of brain ischemia is less than a threshold stage for detecting brain ischemia, the communication circuit 208 may transmit a non-ischemia notification at block 460. In some examples, an identified stage of brain ischemia may be transmitted at one of blocks 460 and 462. Brain ischemia criteria may be met at block 458 when at least one amplitude metric and/or at least one frequency metric correspond to stage 2 or stage 3 or higher.

One or more EEG metrics determined at block 456 may be compared to respective thresholds or ranges for detecting brain ischemia that is evidence of an HU cardiac rhythm at block 458. For example, if an EEG amplitude metric is greater than an applied threshold and/or a frequency metric is less than an applied threshold, brain ischemia criteria may be determined to be met by processing circuit 200 at block 456. A threshold or range applied to a given EEG metric may be stored in memory 202 and can be established based on empirical data from a population of patients or based on a baseline measure of the EEG metric determined from the patient during a normal sinus rhythm, which may be updated over time. EEG sensor communication circuit 206 transmits a notification, and optionally other EEG metric data or brain ischemia stage classification, at block 460 or 462 based on whether brain ischemia criteria are met by the EEG metrics.

In some examples, the rate of onset of an ischemic change in EEG metric(s) may be determined for use in discriminating between HU cardiac rhythms and other conditions that may cause changes in the frequency and amplitude of neural oscillations. For instance, when one or more EEG metrics meet brain ischemia criteria, the time from when the EEG metric(s) do not meet brain ischemia criteria until the time that the EEG metric(s) do meet brain ischemia criteria may be determined when multiple sequential EEG signal episodes are analyzed. The time to onset of an ischemic change in EEG metric(s) may discriminate from other possible causes of EEG changes that are not associated with an HU cardiac rhythm. For example, if one or more EEG metrics already meet brain ischemia criteria prior to or within a threshold time interval from the onset of a concerning heart rate detection, the EEG metrics may be indicative of another condition such as deep sleep, drug effects or other conditions that may mimic the frequency and amplitude characteristics of a brain ischemia stage. In some examples, if the onset of brain ischemia-like EEG metrics appear to be too early relative to the onset of a concerning heart rate detection to be caused by a change in cardiac rhythm, the brain ischemia determination may be unknown. In this case, EEG sensor 40 may transmit an indeterminate or unknown brain ischemia notification at block 460. In some examples, when the brain ischemia is indeterminable due to EEG metrics meeting brain ischemia changes at the onset of the fast heart rate, for example if the patient is asleep when the concerning heart rate is detected, a co-implanted device may determine that a detected VT or VF is HU by default, particularly when a fast heart rate is detected or a tachyarrhythmia is being detected that could be life threatening. For instance, when an EEG signal already having dominant delta band frequency is detected, a co-implanted device may determine that a detected VT or VF is HU by default and select ATP and/or CV/DF therapy delivery accordingly.

Additionally or alternatively, the medical device system may determine sleep based on time of day and/or other sensor input. As described above, one or more implantable device, e.g., EEG sensor 40, cardiac monitor 44, ICD 14, ICD 114, pacemaker 150, may include one or more sensors for sensing another physiological signal, such as patient physical activity, temperature, and/or respiration. Processing circuitry of the medical device system may determine that when brain ischemia criteria are met and the time of day is night, patient physical activity is resting, body temperature is not elevated, respiration rate is low or at a resting level, or any combination thereof, the patient is likely to be asleep. In this case, to avoid a false brain ischemia detection, the medical device system may select a therapy response based on electrical cardiac activity without taking into account electrical brain activity. A notification of indeterminate brain ischemia may be transmitted by EEG sensor 40 at block 460.

Furthermore, it is recognized that in some examples, additional sensors may be available in the medical device system for monitoring other indicators of HU. For example, an implanted medical device may include an oxygen sensor, a blood pressure sensor, a blood volume (e.g., impedance) sensor or other sensor of a physiological signal that may be responsive to changes in hemodynamic stability. Accordingly, when EEG metrics meet brain ischemia criteria but an indeterminate brain ischemia detection is made based on the rate of onset of the brain ischemia and/or detecting a likelihood of sleep, other sensor signals may be analyzed for confirming or rejecting a brain ischemia detection. For example, on oxygen sensor, blood pressure sensor, blood volume sensor (e.g., impedance) or other sensor may be included in the medical device system. When EEG metrics meet brain ischemia criteria and a corroborating change is detected from a secondary sensor signal, e.g., decreased blood or tissue oxygen saturation or decreased blood pressure, brain ischemia may be detected at block 458. When a corroborating change is not detected from the secondary sensor signal, an indeterminate brain ischemia detection or no brain ischemia detection may be made by the medical device system, e.g., by control circuit 80 using information from sensor(s) 89 and a transmitted signal from EEG sensor 40.

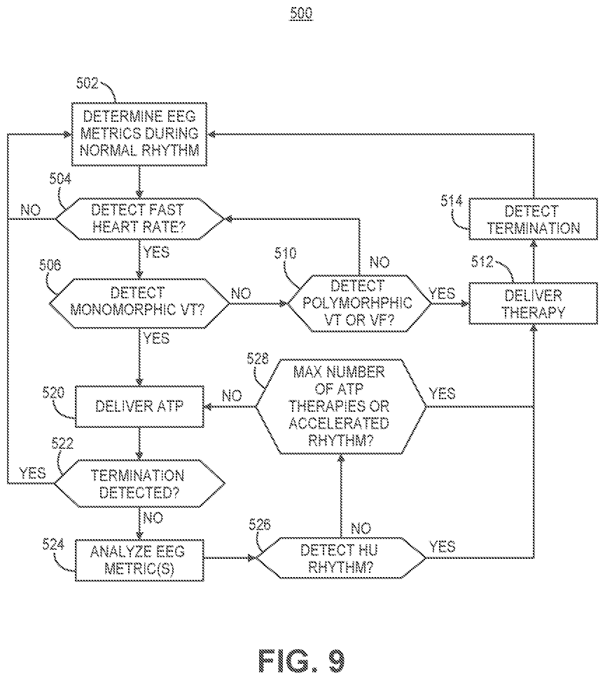

FIG. 9 is a flow chart 500 of a method that may be performed by a medical device system, such as any of the systems shown or described above in conjunction with FIGS. 1-3, for detecting a tachyarrhythmia and selecting a therapy response according to some examples. At block 502, EEG sensor 40 may be configured to determine EEG metrics that are used for detecting brain ischemia during normal sinus rhythm. The normal sinus rhythm may be required to be at a heart rate that is less than a tachyarrhythmia detection rate or less than a specified rate, e.g., less than 100 bpm, and no tachyarrhythmia detection in progress (e.g., the VT and VF interval counters are at zero). EEG metrics may be determined during a normal sinus rhythm and daytime hours when the patient is expected to be awake according to any of the examples described herein. The EEG metrics may be used as baseline metrics for establishing thresholds or other criteria used in detecting a change in the EEG indicative of brain ischemia. The EEG metrics determined at block 502 and/or thresholds or other criteria established by EEG sensor processing circuit 200 based on the determined EEG metrics may be stored in EEG sensor memory 202. When EEG metrics determined at block 502 are used by processing circuit 200 to update baseline metrics, the EEG metrics may first be compared to previous metrics to avoid using new EEG metrics determined during electrical brain activity that does not correspond to normal brain activity or stage 1 brain ischemia, e.g., as shown in FIG. 7.

At block 504, processing circuitry of the medical device system detects a fast heart rate. When a fast heart rate is not being detected (“no” branch of block 504), EEG sensor 40 may re-determine EEG metrics at block 502 on a periodic or scheduled basis when a normal sinus rhythm is being detected for updating baseline values of the EEG metrics and/or update criteria or thresholds for detecting brain ischemia. One or more co-implanted devices may be configured to detect the fast heart rate at block 504. When two or more IMDs of the medical device system are configured to detect a fast heart rate, the two or more IMDs may detect the fast heart rate concurrently at block 504 to enable coordinated detection of a HU or HS cardiac rhythm. For the sake of illustration, the process of flow chart 500 is described as being performed by EEG sensor 40 co-implanted with ICD 14 (shown in FIG. 1). ICD control circuit 80 may detect a fast heart rate at block 504 and may transmit a request to EEG sensor 40 to begin determining EEG metrics in some examples so that changes and the time course over which any changes occur in EEG metrics can be determined. In other examples, EEG sensor 40 may extract ECG signals from the signal sensed via electrodes 42 and detect a fast heart rate at block 504, which may be concurrent with a fast heart rate detection by ICD 14. EEG sensor 40 may begin buffering EEG signal episodes when a fast heart rate is detected, e.g., greater than 140, 160, 180 or 200 bpm, so that EEG metrics can be determined and available if the hemodynamic status of the cardiac rhythm is needed by ICD 14 for making a therapy delivery selection.

At block 506, ICD control circuit 80 may detect a monomorphic VT. For example, when RRIs determined from a sensed ECG signal reach an NID for detecting VT, ICD control circuit 80 may analyze the RRIs for regularity. RRIs tend to be more regular and can occur at slower rates (longer RRIs) during monomorphic VT than during polymorphic VT or VF. SVTs can sometimes be distinguished from monomorphic VTs based on irregularity of the RRIs as the atrial depolarizations can be irregularly conducted to the ventricles during SVT. An example technique for detecting a monomorphic VT based on RRI analysis is generally disclosed in U.S. Pat. No. 9,808,637 (Sharma, et al.), incorporated herein by reference in its entirety.

Additionally or alternatively, monomorphic VT may be discriminated from polymorphic VT or VF (and in some cases SVT) by ICD control circuit 80 at block 506 based on ECG (or EGM) morphology analysis. ICD control circuit 80 may compare the waveform morphology of sensed signals to each other or to an R-wave template stored in memory 82 and/or analyze other sensed waveform features for discriminating between monomorphic VT and polymorphic VT or VF. Example techniques for discriminating between monomorphic and polymorphic tachyarrhythmias may include determining the frequency content of sensed QRS waveforms, determining one or more slopes of the QRS waveform, determining morphology matching scores between sensed QRS waveforms and/or between sensed QRS waveforms and an R-wave template stored in memory 82.

Example techniques for discriminating between polymorphic and monomorphic tachyarrhythmias are generally disclosed in U.S. Pat. No. 7,076,289 (Sarkar, et al.) and U.S. Pat. No. 7,130,677 (Brown, et al.), both of which are incorporated herein by reference in their entirety. The techniques disclosed herein for determining whether a detected tachyarrhythmia is HU or HS are not limited to any particular tachyarrhythmia detection and discrimination techniques. A wide variety of techniques for detecting and discriminating between monomorphic VT, polymorphic VT or VF, and SVT may be employed in conjunction with the techniques disclosed herein.

When ICD control circuit 80 detects monomorphic VT at block 506, control circuit 80 may control therapy delivery circuit 84 to deliver ATP at block 520. After the first delivered sequence of ATP pulses, ICD control circuit 80 determines if termination of the VT is detected at block 522. Termination may be detected based on a threshold number of RRIs being greater than the tachycardia detection threshold, for example. When termination of the monomorphic VT is detected following ATP delivery, the process may return to block 502. ICD control circuit 80 may continue to monitor RRIs for detecting a fast heart rate at block 504. EEG sensor 40 may update EEG metrics during a normal, sinus rhythm at block 502 as described above.

When termination of the VT is not detected at block 522 following the first ATP sequence, EEG metrics may be analyzed at block 524 for determining if the detected VT is HU or HS. ICD 14 may transmit a communication signal to EEG sensor 40 requesting a determination of whether brain ischemia based on EEG metrics is detected as evidence of an HU VT. EEG sensor 40 may buffer one or more EEG signal episodes and determine EEG metrics according to any of the examples described above, e.g., in conjunction with FIGS. 7 and 8. EEG sensor 40 may analyze the EEG metrics at block 524 by comparing the EEG metric(s) to brain ischemia criteria. In some examples, EEG sensor 40 may have already buffered one or more EEG signal episodes and determined EEG metrics from each buffered episode for comparison to brain ischemia criteria at block 524 at the time of receiving a request from ICD 14, for example when EEG sensor 40 is configured to monitor the EEG signal on a continuous or intermittent basis or is configured to detect a fast heart rate for triggering EEG signal analysis. In other examples, EEG sensor 40 starts EEG signal episode buffering and EEG metric determination in response to receiving a request from ICD 14 transmitted by ICD communication circuit 88 when a fast heart rate is detected at block 504, when monomorphic VT is detected at block 506 or when termination is not detected at block 522 after the first ATP therapy. As indicated above, in some instances, EEG sensor 40 may start buffering an EEG signal episode after a specified time delay to account for a lag between the onset of brain ischemia and the onset or time of detection of a fast heart rate or VT.

EEG sensor 40 may determine if EEG metrics meet brain ischemia criteria, which may include determining a stage of brain ischemia based on determined EEG metrics as described in various examples given above. If brain ischemia criteria are not met, which may include determining stage 1 (or in some examples stage 2) of brain ischemia as described in examples given above, EEG sensor 40 may transmit a notification of no brain ischemia (or a notification of an HS cardiac rhythm) to ICD 14. In response to receiving the notification of no brain ischemia, ICD 14 may detect an HS VT at block 526 (“no” branch”). ICD 14 may return to block 520 to deliver another ATP therapy if the VT is still being detected. When the detected VT is determined to be HS at block 526, multiple sequences of ATP may be delivered at block 520 if termination is not detected at block 522 after the preceding ATP sequence and the detected VT continues to be determined as HS at block 526. A clinician may program a menu of ATP therapies to be delivered in series when termination is not detected following a previous ATP therapy, and the detected VT has not accelerated and/or is not HU. In some examples, control circuit 80 may adjust the interval(s) between ATP pulses, e.g., to provide a sequence of ATP pulses at shorter ATP intervals than in a previous ATP sequence, when the detected VT is not terminated by a delivered ATP therapy.

Accordingly, when the detected VT is identified as HS at block 526 (“no” branch), ICD control circuit 80 may determine if additional ATP therapies remain in a programmed menu or series of ATP therapies at block 528. ICD control circuit 80 may verify that the detected ventricular rate has not accelerated, e.g., by determining that the most recent RRIs determined after ATP delivery are not shorter than a fast VT or VF threshold interval. If all ATP therapies have been delivered in a programmed series of ATP therapies or maximum number of ATP therapy attempts have been reached, or if the ventricular rate is accelerated since the initial monomorphic VT detection, ICD control circuit 80 may advance to block 512 to deliver one or more CV/DF shocks for terminating the tachyarrhythmia. As indicated above in conjunction with FIG. 7, if the amplitude and/or frequency metric(s) determined from the sensed electrical brain activity meet severe brain ischemia criteria (e.g., corresponding to stage 5 when suppression of neural oscillations occurs) after detecting an HU cardiac rhythm associated with a fast heart rate, CV/DF shocks (and any other therapy) may be cancelled or aborted in some examples, particularly if one or more shocks have already been attempted and/or stage 5 brain ischemia is detected for a threshold period of time, e.g., several minutes.

Referring again to block 506, if monomorphic VT is not detected at block 506, ICD control circuit 80 may determine if polymorphic VT or VF is detected at block 510. In some instances the fast heart rate may be an SVT, e.g., normal sinus tachycardia or conducted atrial tachyarrhythmia, such as rapidly conducted AF. In some examples, fast VT or VF may be detected at block 510 based on the sensed ventricular rate, e.g., based on RRIs being shorter than a fast VT or VF interval threshold. In some examples, an SVT limit may be applied such that RRIs shorter than the SVT limit (and a fast VT or VF detection interval) may result in a polymorphic VT or VF detection at block 510. Interval and morphology based methods for discriminating between polymorphic VT or VF and monomorphic VT are generally disclosed in the above-incorporated U.S. Pat. No. 9,808,637 (Sharma, et al.), U.S. Pat. No. 7,076,289 (Sarkar, et al.), U.S. Pat. No. 7,130,677 (Brown, et al.). Further, SVT may optionally be discriminated from VT or VF based on rate, irregularity of RRIs and/or morphology matching scores between sensed QRS waveforms and an R-wave template as examples. Example techniques for discriminating between SVT and VT/VF are generally disclosed in U.S. Pat. No. 9,675,261 (Cao, et al.), incorporated herein by reference in its entirety. Other example techniques for rejecting a VT/VF detection based on evidence of an SVT are generally disclosed in U.S. Pat. No. 10,555,684 (Zhang et al.), incorporated herein by reference in its entirety.

If polymorphic VT or VF is not detected at block 510 (“no” branch), ICD control circuit 80 may return to block 504 and continue monitoring RRIs. When a polymorphic VT or VF is detected at block 510, ICD control circuit 80 may control therapy delivery circuit to deliver therapy at block 512 without necessarily requiring a determination of whether the rhythm is HS or HU based on EEG signals. Therapy delivery circuit 84 may generate and deliver one or more CV/DF shocks until termination is detected at block 514. ATP may be delivered before CV/DF shock in some instances, e.g., when a programmed sequence of therapies to be delivered in response to VT/VF detection includes one or more sequences of ATP therapies prior to CV/DF shock therapies. In some cases, ATP may be delivered during high voltage capacitor charging in preparation of CV/DF shock delivery. When termination is detected at block 514 following a delivered therapy, the process may return to block 502. It is to be understood that in some instances, a non-sustained tachyarrhythmia may occur. ICD control circuit 80 may be configured to detect spontaneous termination of a detected VT or VF prior to therapy delivery, e.g., during high voltage capacitor charging, and cancel a pending therapy and return to block 502.

In the example of flow chart 500, a determination of HU or HS VT is made by the medical device system when a monomorphic VT is detected and is not terminated after an initial ATP therapy attempt. However, it is to be understood that in other examples, the determination of an HU or HS tachyarrhythmia may be made at any time during the process of flow chart 500 to guide therapy delivery selection and timing. For example, EEG sensor 40 may be configured to buffer EEG signal episodes in a rolling manner for monitoring for evidence of brain ischemia based on EEG metrics, which may be independent of the timing or type of cardiac rhythm being detected. EEG sensor 40 may transmit a signal indicating whether brain ischemia is detected or not. As long as brain ischemia is not being detected, therapy delivery circuit 84 may be controlled to select a therapy response, which may include delivering one type of therapy, e.g., ATP while withholding or delaying another type of therapy delivery (e.g., withhold CV/DF shocks). ICD 14 (or ICD 114 or pacemaker 150) may continue delivering ATP until the tachyarrhythmia is terminated or an HU VT is detected based on a detection of brain ischemia.

Furthermore, while the process of flow chart 500 is described in conjunction with a medical device system including co-implanted ICD 14 and EEG sensor 40, it is to be understood that in other examples, cardiac monitor 40 may be co-implanted with ICD 14 and EEG sensor 40 and processing circuitry of cardiac monitor 40 may perform processing and analysis for detecting and discriminating cardiac rhythms and communicating with EEG sensor 40 and/or ICD 14 for cooperatively detecting HU or HS tachyarrhythmia and selecting and controlling the therapy delivery response. In still other examples, pacemaker 150 may be included in the medical device system and may be configured to deliver ATP therapy when ATP is being delivered to treat HS monomorphic VT or prior to a CV/DF shock for treating an HU VT/VF. Furthermore, if an ICD is not co-implanted for delivering CV/DF shocks, an implanted pacemaker, such as pacemaker 150, may be configured to deliver ATP when HU VT is detected. In other examples, the process of flow chart 600 may be performed by a medical device system that includes an ICD connected to transvenous leads, e.g., ICD 114 as shown in FIG. 2, in cooperation with EEG sensor 40.

FIG. 10 is a flow chart 600 of a method that may be performed by a medical device system, such as any of the systems shown or described above in conjunction with FIGS. 1-3, for detecting a tachyarrhythmia and selecting a therapy response according to another example. Some tachyarrhythmia detection algorithms may include “gray” zones or overlapping criteria that may be satisfied during different cardiac rhythms. For example, some criteria applied to a sensed cardiac electrical signal may be met during both SVT and monomorphic VT or during both monomorphic VT and fast VT/VF. For instance, the RRIs that occur during SVT may overlap with RRIs that occur during monomorphic VT. The RRIs that occur during monomorphic VT may overlap with the RRIs that can occur during polymorphic VT. In another example, a morphology matching score determined between a sensed QRS waveform and an R-wave template may be very high, e.g., greater than 70%, for confirming an SVT, or very low, e.g., less than 30% for confirming VF with a high degree of confidence. At times, however, a morphology matching score may fall somewhere in between in a so-called “gray” zone in which the morphology matching score may not provide discrimination between SVT, VT and VF with a high degree of confidence. Example tachyarrhythmia detection techniques that include morphology matching score gray zones are generally disclosed in U.S. Pat. No. 8,437,842 (Zhang, et al.), incorporated herein by reference in its entirety. A tachyarrhythmia detection may be made with relatively low or high confidence depending on whether various parameters determined from a cardiac electrical signal for comparison to tachyarrhythmia detection criteria fall into a confident detection zone or a gray zone.

Additionally or alternatively, some tachyarrhythmia detection algorithms may include oversensing analysis to minimize the likelihood of detecting a tachyarrhythmia due to oversensing electrical noise or other signals as false R-waves. For example, a fast heart rate may be falsely detected due to oversensing of non-cardiac noise such as skeletal muscle myopotentials or electromagnetic interference, cross-chamber oversensing of P-waves as false R-waves, or T-wave oversensing. When possible oversensing of noise or other signals is identified based on cardiac electrical signal analysis, the confidence in detecting a VT or VF based on other tachyarrhythmia detection criteria being met, such as NID, may be reduced.

Some tachyarrhythmia detection algorithms may rely on AI models or probability models that can output a rhythm classification with an associated confidence level, e.g., as a percentage, or overall probability. In a tachyarrhythmia detection model, a relatively low confidence level or probability may be output when a rhythm classification is SVT, VT or VF. Example methods for detecting a tachyarrhythmia using a probability-based model are generally disclosed in U.S. Pat. No. 8,301,233 (Zhang et al.) and in U.S. Publication No. 2020/0038671 (Schulhauser et al.), both of which are incorporated herein by reference in their entirety. Some examples of AI techniques that may be used in a medical device system for classification of an episode of arrhythmia are generally disclosed in U.S. Publication No. 2020/0357519 (Chakravarthy, et al.), incorporated herein by reference in it is entirety.

Accordingly, depending on the particular tachyarrhythmia detection techniques implemented in an IMD co-implanted with EEG sensor 40, an SVT, VT or VF detection may be made with varying levels of confidence or probability. A medical device system operating according to techniques disclosed herein may analyze EEG metrics for detecting evidence of brain ischemia when a fast heart rate is detected but a tachyarrhythmia detection confidence or probability level is relatively low. A threshold confidence level may be programmable or predefined depending on the particular tachyarrhythmia detection techniques being deployed. For example, a tachyarrhythmia detection may be determined to be highly confident when the probability or confidence level is at least 60%, at least 70%, at least 80% or at least 90%.

In flow chart 600, at block 602, the EEG sensor 40 may establish or update baseline EEG metrics for a given patient during a normal sinus rhythm as generally described above. EEG metric thresholds or other criteria for detecting brain ischemia or identifying different stages of brain ischemia may be established based on the baseline EEG metrics determined from the patient. In other examples, the EEG metric threshold(s) or other criteria applied for detecting brain ischemia or identifying different stages of brain ischemia may be programmed by a user or previously established based on empirical data and stored in memory 202 of EEG sensor 40.

For the sake of illustration, FIG. 10 is described in conjunction with ICD 14 and EEG sensor 40 being co-implanted and in communication with each other for cooperatively detecting tachyarrhythmia and identifying the tachyarrhythmia as HU or HS. However, it is to be understood that other combinations of medical devices as described in any of the examples given above may be configured to perform the method of flow chart 600, wholly or in part.

At block 604 the ICD 14 may detect a fast heart rate. When a fast heart rate is detected, a tachyarrhythmia detection algorithm may begin or be in progress, which may include counting VT and VF intervals, morphology analysis of cardiac electrical signal segments, or determination of other parameters of electrical cardiac activity for detecting tachyarrhythmia from one or more cardiac electrical signals sensed by ICD 14. It is to be understood, however, that detection of a fast heart rate at block 604 may be optional in some examples. For instance, cardiac electrical signal segments may be analyzed for classification according to a cardiac rhythm type without necessarily relying on interval or rate-based criteria that requires detection of a fast heart rate based on RRIs.

At block 606, ICD control circuit 80 may detect SVT with a high confidence. For example, SVT may be detected with high confidence when the ventricular rate (as determined from RRIs) is slower than a fast VT or VF threshold rate (or an SVT limit) but faster than a VT threshold rate and one or more QRS waveform morphology matching scores are greater than a confident SVT match threshold. In other probabilistic models, the confident SVT detection at block 606 may be made when a confidence level of a cardiac rhythm classification model is greater than a high confidence threshold, e.g., greater than 70% or greater than 80%. When SVT is detected with high confidence, the ICD control circuit 80 may determine that no therapy is to be delivered at block 608. During an SVT, cardiac output may be sufficient to maintain CBF at normal or near normal levels such that no changes in the EEG are expected. As such EEG signal analysis may not be performed by the medical device system when an SVT is detected with high confidence.

In other examples, however, it is contemplated that when SVT is detected at block 606, the process may advance to block 624 to determine EEG metrics for identifying the cardiac rhythm as being HU or HS. In some cases, an SVT may be conducted irregularly to the ventricles or be occurring at a very fast rate that results in an HU cardiac rhythm.

When SVT is not detected or not detected with high confidence (“no” branch of block 606), the ICD control circuit 80 may detect monomorphic VT with high confidence at block 610. If monomorphic VT is detected with high confidence, control circuit 80 may control therapy delivery circuit 84 to deliver ATP at block 620. When termination is detected following ATP delivery (block 622) the process of flow chart 600 may return to block 602. If termination is not detected following ATP, or if undersensing of R-waves is suspected, EEG sensor 40 may determine EEG metrics for comparison to brain ischemia criteria at block 624. Undersensing may be suspected when the peak amplitude of sensed R-waves is near a programmed sensitivity such that some R-waves may be undersensed. As generally described above, EEG sensor 40 may receive a communication signal from ICD 14 requesting EEG analysis for detecting brain ischemia as evidence of an HU tachyarrhythmia. In other examples, EEG sensor 40 may be configured to detect the fast heart rate and begin monitoring the EEG signal for detecting brain ischemia based on EEG metrics according to any of the examples described herein. EEG sensor 40 may transmit a determination of brain ischemia (or no ischemia) for receipt by ICD 14.

If ICD 14 does not receive an indication of brain ischemia from EEG sensor 40, control circuit 80 may determine that the detected VT is not HU at block 626. Control circuit 80 may control therapy delivery circuit 84 to deliver another ATP therapy at block 628. As described above, another ATP therapy may be delivered in a series or menu of programmed ATP therapies until all ATP therapies have been delivered or until termination is detected at block 622. While not shown explicitly in FIG. 10, it is to be understood that an accelerated rhythm may be detected by control circuit 80 after an ATP therapy in which case control circuit 80 may control therapy delivery circuit 84 to deliver a CV/DF shock as generally described above in conjunction with FIG. 9. Referring again to block 624, when ICD 14 receives a notification of brain ischemia from EEG sensor 40, control circuit 80 detects an HU tachyarrhythmia at block 626 and advances to block 614 to deliver a CV/DF shock (which may be preceded by ATP in some cases, e.g., during high voltage charging).

Referring again to block 610, when monomorphic VT is not detected or not detected with a high confidence, control circuit 80 may detect a polymorphic VT or VF at block 612. If the polymorphic VT or VF is detected with high confidence, control circuit 80 may control therapy delivery circuit 84 to deliver CV/DF shock therapy at block 614 (which may be preceded by ATP therapy if ATP sequences are programmed in a menu of therapies and/or when ATP therapy can be delivered during high voltage capacitor charging). One or more therapies may be delivered at block 614 for terminating a polymorphic VT or VF detected with high confidence without necessarily determining and analyzing EEG metrics. When termination is detected at block 616, the process may return to block 602.

If a polymorphic VT or VF is not detected at block 612 with high confidence, the medical device system may determine EEG metrics for comparison to brain ischemia criteria at block 624. In some examples, when SVT, VT or VF is detected with low confidence, the EEG signal may be analyzed for determining if the cardiac rhythm is HU or HS. If the EEG metrics meet brain ischemia criteria, the cardiac rhythm may be detected as being HU at block 626. ICD control circuit 80 may advance to block 614 for delivering CV/DF therapy. If the EEG metrics do not meet brain ischemia criteria, therapy delivery circuit 84 may deliver ATP at block 628. In other examples, control circuit 80 may withhold all therapy if brain ischemia criteria are not met, indicating an HS cardiac rhythm. An ongoing fast rate may be an HS SVT that does not necessarily require any therapy. The therapy decision made when brain ischemia criteria are not met may depend on the cardiac rhythm detected with low confidence. For instance, if an SVT is detected with low confidence and brain ischemia criteria are not met, all ATP and CF/DF shock therapy may be withheld. If monomorphic VT is detected with low confidence and brain ischemia criteria are not met, ATP may be delivered and CV/DF shock may be withheld or delayed. After delivering (or withholding ATP), the process may return to block 622 and continue monitoring the cardiac electrical signal(s) for detecting termination of the fast heart rate or tachyarrhythmia. As long as termination is not detected at block 622, EEG metrics may be determined from EEG signal episodes (block 624) to detect if the cardiac rhythm becomes HU (block 626).

Furthermore, EEG metrics may continue to be detected and monitored for a specified time interval after termination of a tachyarrhythmia is detected to verify restoration of hemodynamic stability. In some instances, a VT/VF may be undersensed, e.g., due to low amplitude R-waves or fibrillation waves. Therefore termination may be falsely detected. By continuing EEG signal analysis after a termination detection, the medical device system may verify that a HS cardiac rhythm is restored.

FIG. 11 is a flow chart of a method 700 for detecting HU and HS cardiac rhythms according to another example. Blocks 302 through 322 in FIG. 11 may generally correspond to identically numbered blocks shown in FIG. 6 and described above. In some examples, when EEG metrics meet brain ischemia criteria at block 306, processing circuitry of the medical device system may be configured to determine if a concerning heart rate is still being detected at block 702. If not, the concerning heart rate may have spontaneously terminated. However, in some cases, a false termination of a concerning heart rate may be detected. For instance, when P-waves, T-waves, noise or other artifacts are oversensed as false R-waves a slow heart rate may be falsely determined to be terminated due to oversensing. In other instances, when true R-waves are undersensed, e.g., during VT or VF, a fast heart rate may be falsely determined to be terminated due to undersensing. When the EEG metrics meet brain ischemia criteria but a concerning heart rate is no longer detected at block 702, the medical device system processing circuitry may continue to monitor the EEG metrics to verify that recovery from the brain ischemia, indicating restoration of an HS cardiac rhythm, occurs.

In some examples, ICD control circuit 80 may be configured to adjust a control parameter used in detecting the concerning heart rate to avoid oversensing when a slow heart rate is no longer being detected or to avoid undersensing when a fast heart rate is no longer being detected. For example, control circuit 80 may adjust parameters used to control an R-wave sensing threshold (such as the ventricular sensitivity, one or more decay rates or times, one or more drop time intervals or step decrements or any other parameter used to control an auto-adjusting R-wave sensing threshold). Additionally or alternatively, other R-wave sensing control parameters may be adjusted such as a ventricular blanking period or refractory period. Additionally or alternatively, control circuit 80 may adjust VT/VF detection parameters to be more sensitive to detecting VT/VF when the EEG metrics meet brain ischemia criteria, particularly if brain ischemia recovery is not detected after the concerning heart rate is no longer being detected.

The adjustment(s) at block 704 may be temporary. Cardiac event signal sensing and/or arrhythmia detection control parameters that are adjusted at block 704 may be restored to previously programmed settings after a specified period of time or if termination of the concerning heart rate is still detected at block 312 after making the adjustments. If termination of the concerning heart rate is not detected, EEG metrics may continue to be monitored by returning to block 304 (“no” branch of block 312) until termination of the concerning heart rate is detected, which may include one or more adjustments to sensing and/or detection control parameters at block 704. In some examples, adjustment of control parameters used in detecting a concerning heart rate from electrical cardiac activity at block 704 may include determining if undersensing or oversensing of cardiac electrical event signals, e.g., R-waves, is suspected. When undersensing or oversensing of cardiac electrical event signals is suspected, e.g., based on sensed event interval (e.g., RRIs) and/or peak amplitudes, the ICD control circuit 80 may detect termination of the concerning rhythm at block 312.

In some instances EEG metrics may continue to meet brain ischemia criteria after the concerning heart rate is no longer detected. The brain ischemia may be falsely detected or may be caused by other conditions, other than an HU cardiac rhythm. In this case, therapy delivery for treating an HU cardiac rhythm may be appropriately withheld. For example, when brain ischemia criteria are still met after a concerning heart rate is no longer detected, even after making adjustments for detecting cardiac electrical activity or accounting for undersensing or oversensing, may be caused by a stroke or other patient condition that cannot be remedied by delivering an electrical stimulation therapy. In this case, however, it is to be understood that the processing circuitry of the medical device system may generate an output for transmitting an alert to call EMS, alert a clinician or other caregiver, and/or alert the patient. Medical attention may be required and/or reprogramming of brain ischemia detection and/or concerning heart rate detection control parameters may be warranted.

In some examples, when the EEG metrics meet brain ischemia criteria at block 306 and the concerning heart rate is still being detected at block 702, the processing circuitry of the medical device system may determine if sleep criteria are met at block 706. Control circuit 80 may detect sleep based on at least one of the time of day and/or an available sensor signal received by the processing circuitry, e.g., according to any of the examples given above. For instance, if the time of day is night, patient posture is non-upright, patient physical activity is resting, brain ischemia onset is too early or slow relative to the time of detection of the concerning heart rate, respiration rate is less than a threshold resting rate, or any combination thereof, sleep criteria may be determined to be met by ICD control circuit 80 at block 706.

In other examples, ICD control circuit 80 may generate an arousal signal at block 706, e.g., by causing a buzzing of an accelerometer in the ICD 14 or generating an audible sound from a microphone, to cause arousal of the patient if the patient is sleeping. A change in the EEG metric(s) following an arousal signal may be evidence that the EEG metrics meeting brain ischemia criteria may be a false ischemia detection due to the patient being asleep. In this case, ICD control circuit 80 may determine that sleep criteria are met (“yes” branch of block 706) if the EEG metric(s) change after the arousal signal (e.g., brain ischemia criteria no longer met or a brain ischemia stage decreases toward stage 1 or 2). If sleep criteria are not met, e.g., if the EEG metric(s) do not change after the arousal signal, control circuit 80 may detect an HU cardiac rhythm at block 320 based on detecting brain ischemia from the electrical brain activity sensed after the arousal signal. The processing circuitry may generate an output at block 322 for providing a response to the HU cardiac rhythm at block 322 as generally described above.

If sleep criteria are met at block 706, the medical device system processing circuitry may optionally determine if another sensor signal is available for confirmation of an HU cardiac rhythm at block 708. For example, when an oxygen sensor, blood pressure sensor or other physiological sensor signal responsive to hemodynamic changes is received by the medical device processing circuitry, the sensor signal may be analyzed for detecting a change that may be indicative of an HU cardiac rhythm. If another sensor signal is available that confirms a likelihood of an HU cardiac rhythm, e.g., decreased oxygen saturation, decreased blood pressure or the like, control circuit 80 may advance to block 320 to detect an HU cardiac rhythm.

If another sensor signal is not available or does not indicate an HU cardiac rhythm, (e.g., no decrease in blood pressure or normal oxygen saturation), the processing circuitry, e.g., ICD control circuit 80, may select a therapy based on the sensed cardiac electrical activity at block 710, ignoring the sensed electrical brain activity. EEG metrics meeting the brain ischemia criteria may be a false positive, e.g., when the patient is asleep or due to other physiological conditions. As such, control circuit 80 may select a therapy response at block 710 based on electrical cardiac activity and not based on the electrical brain activity when sleep criteria are met at block 706. The cardiac electrical activity may be a slow heart rate that requires cardiac pacing or a fast heart rate that requires ATP and/or CV/DF therapy. The process may advance to block 324 to determine if the concerning heart rate is terminated and may continue monitoring the EEG metric(s) if the concerning heart rate is still being detected, as generally described above in conjunction with FIG. 6.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device system has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device system comprising:
sensing circuitry configured to sense electrical brain activity and electrical cardiac activity;
processing circuitry configured to:
detect a concerning heart rate from the sensed electrical cardiac activity;
determine that the concerning heart rate is a hemodynamically unstable cardiac rhythm based on the sensed electrical brain activity; and
generate an output comprising at least a therapy control signal for providing a response to determining that the concerning heart rate is a hemodynamically unstable cardiac rhythm;
a memory configured to store the output generated by the processing circuitry; and
a therapy delivery circuit configured to deliver an electrical stimulation therapy according to the therapy control signal.

2. (canceled)

3. The medical device system of claim 1 wherein the processing circuitry is further configured to:
determine at least one amplitude metric from the electrical brain activity;
compare the at least one amplitude metric to brain ischemia criteria; and
determine that the concerning heart rate is a hemodynamically unstable cardiac rhythm in response to the at least one amplitude metric meeting the brain ischemia criteria.

4. The medical device system of claim 1, wherein the processing circuitry is further configured to:
determine at least one frequency metric from the electrical brain activity by determining at least one of:
a dominant frequency of the electrical brain activity;
a number of oscillations of the electrical brain activity during a time interval; and
an amplitude of a signal derivative of the electrical brain activity;
compare the at least one frequency metric to brain ischemia criteria;
determine that the at least one frequency metric meets brain ischemia criteria; and
determine that the concerning heart rate is a hemodynamically unstable cardiac rhythm in response to the at least one frequency metric meeting the brain ischemia criteria.

5. The medical device system of claim 1 wherein the processing circuitry is further configured to:
determine at least one metric of the electrical brain activity;
determine a stage of brain ischemia from a plurality of stages of brain ischemia based on the at least one metric; and
determine that the concerning heart rate is a hemodynamically unstable cardiac rhythm based on the stage of brain ischemia.

6. The medical device system of claim 1 wherein the processing circuitry is further configured to:
determine a metric from the electrical brain activity for each of a plurality of time intervals;
determine that a change in the metric determined for each of the plurality of time intervals meets brain ischemia criteria; and
determine that the concerning heart rate is a hemodynamically unstable cardiac rhythm based on the change in the metric meeting brain ischemia criteria within a threshold time interval of an onset of the concerning heart rate.

7. (canceled)

8. The medical device system of claim 1 wherein the therapy delivery circuit is configured to deliver a cardioversion/defibrillation shock, and wherein:
the processing circuitry is further configured to:
detect the concerning heart rate by detecting a heart rate that is faster than a threshold rate;
determine that the sensed electrical brain activity meets severe brain ischemia criteria; and
control the therapy delivery circuit to abort a cardioversion/defibrillation shock in response to determining that the sensed electrical brain activity meets severe brain ischemia criteria.

9. The medical device system of claim 1 wherein the processing circuitry is further configured to:
detect a monomorphic ventricular tachyarrhythmia associated with the concerning heart rate based on the sensed cardiac electrical activity;
control the therapy delivery circuit to deliver a first anti-tachycardia pacing therapy in response to detecting the monomorphic ventricular tachyarrhythmia;
determine that the monomorphic ventricular tachyarrhythmia is not terminated by the first anti-tachycardia pacing therapy;
in response to determining that the monomorphic ventricular tachyarrhythmia is not terminated, determine at least one metric from the sensed electrical brain activity;
determine that the concerning heart rate is a hemodynamically unstable cardiac rhythm based on the sensed electrical brain activity by determining that the monomorphic ventricular tachyarrhythmia is a hemodynamically unstable cardiac rhythm based on the at least one metric; and
generate the output for providing the response by generating a cardioversion/defibrillation shock therapy signal.

10. (canceled)

11. The medical device system of claim 1 wherein the processing circuitry is further configured to:
detect the concerning heart rate by detecting one of a heart rate slower than a threshold rate or a heart rate that meets variable heart rate criteria; and
generate the output by one or more of generating a cardiac pacing delivery signal or increasing a lower pacing rate.

12. The medical device system of claim 1 further comprising a communication circuit,
wherein the processing circuitry is further configured to generate the output for providing the response by generating an alert signal; and
the communication circuit is configured to transmit the alert signal.

13. The medical device system of claim 1 further comprising at least one sensor configured to sense a sensor signal received by the processing circuitry;
wherein the processing circuitry is further configured to:
detect sleep based on at least one of a time of day or the sensor signal;
determine that the concerning heart rate is hemodynamically unstable is indeterminate based on the sensed electrical brain activity in response to detecting sleep.

14. The medical device system of claim 1 further comprising at least one sensor configured to sense a sensor signal received by the processing circuitry;
the processing circuitry being further configured to:
detect sleep based on at least one of a time of day or the sensor signal;
generate an arousal signal;
detect brain ischemia based on the electrical brain activity sensed after the arousal signal; and
determine that the concerning heart rate is a hemodynamically unstable cardiac rhythm based on detecting the brain ischemia based on the electrical brain activity sensed after the arousal signal.

15. The medical device system of claim 1 wherein the processing circuitry is further configured to:
detect termination of the concerning heart rate;
determine that the sensed electrical brain activity still meets brain ischemia criteria after detecting termination of the concerning heart rate; and
adjust a control parameter used in detecting the concerning heart rate from the sensed electrical cardiac activity in response to determining that the sensed electrical brain activity still meets brain ischemia criteria.

16. A method comprising:
sensing electrical brain activity;
sensing electrical cardiac activity;
detecting a concerning heart rate from the sensed electrical cardiac activity;
determining that the concerning heart rate is a hemodynamically unstable cardiac rhythm based on the sensed electrical brain activity;
generating an output comprising at least a therapy control signal for providing a response to determining that the concerning heart rate is a hemodynamically unstable rhythm;
storing the generated output in a memory; and
delivering an electrical stimulation therapy according to the therapy control signal.

17. The method of claim 16 further comprising:
determining at least one amplitude metric from the electrical brain activity;
comparing the at least one amplitude metric to brain ischemia criteria; and
determining that the concerning heart rate is a hemodynamically unstable cardiac rhythm in response to the at least one amplitude metric meeting the brain ischemia criteria.

18. The method of claim 16 further comprising:
determining at least one frequency metric from the electrical brain activity by determining at least one of:
a dominant frequency of the electrical brain activity;
a number of oscillations of the electrical brain activity during a time interval; and
an amplitude of a signal derivative of the electrical brain activity;
comparing the at least one frequency metric to brain ischemia criteria;
determining that the concerning heart rate is a hemodynamically unstable cardiac rhythm in response to the at least one frequency metric meeting the brain ischemia criteria.

19. The method of claim 16 further comprising:
determining at least one metric of the electrical brain activity;
determining a stage of brain ischemia from a plurality of stages of brain ischemia based on the at least one metric; and
determining that the concerning heart rate is a hemodynamically unstable cardiac rhythm based on the stage of brain ischemia.

20. The method of claim 16 further comprising:
determining a metric from the electrical brain activity for each of a plurality of time intervals;
determining that a change in the metric determined for each of the plurality of time intervals meets brain ischemia criteria; and
determining that the concerning heart rate is a hemodynamically unstable cardiac rhythm based on the change in the metric meeting brain ischemia criteria within a threshold time interval of an onset of the concerning heart rate.

21. The method claim 16 further comprising:
detecting a monomorphic ventricular tachyarrhythmia associated with the concerning heart rate based on the sensed cardiac electrical activity;
delivering a first anti-tachycardia pacing therapy in response to detecting the monomorphic ventricular tachyarrhythmia;
determining that the monomorphic ventricular tachyarrhythmia is not terminated by the first anti-tachycardia pacing therapy;
in response to determining that the monomorphic ventricular tachyarrhythmia is not terminated, determining at least one metric from the sensed electrical brain activity;
determining that the concerning heart rate is a hemodynamically unstable cardiac rhythm based on the sensed electrical brain activity by determining that the monomorphic ventricular tachyarrhythmia is a hemodynamically unstable cardiac rhythm based on the at least one metric; and
generating the output for providing the response by generating a cardioversion/defibrillation shock therapy signal.

22. The method of claim 16 further comprising:
detecting the concerning heart rate by detecting one of a heart rate slower than a threshold rate or a heart rate that meets variable heart rate criteria; and
generating the therapy control signal by one or more of generating a cardiac pacing delivery signal or increasing a lower pacing rate.

23. The method of claim 16 further comprising:
sensing a sensor signal;
detecting sleep based on at least one of a time of day or the sensor signal;
generating an arousal signal;
detecting brain ischemia based on the electrical brain activity sensed after the arousal signal; and
determining that the concerning heart rate is a hemodynamically unstable cardiac rhythm based on detecting the brain ischemia based on the electrical brain activity sensed after the arousal signal.
